SEQUENCE LISTING (14 sequences)

SEQ ID NO: 1 - protein, 180 aa, Homo sapiens
MGIPMGKSMLVLLTFLAFASCCIAAYRPSETLCGGELVDTLQFVCGDRGFYFSRPASRVSRRSRGIVEECCFRSCDLALLETYCATPAKSERDVSTPPTVLPDNFPRYPVGKFFQYDTWKQSTQRLRRGLPALLRARRGHVLAKELEAFREAKRHRPLIALPTQDPAHGGAPPEMASNRK

SEQ ID NO: 2 - DNA, 540 bp, Homo sapiens
atgggaatcccaatggggaagtcgatgctggtgcttctcaccttcttggccttcgcctcgtgctgcattgctgcttaccgccccagtgagaccctgtgcggcggggagctggtggacaccctccagttcgtctgtggggaccgcggcttctacttcagcaggcccgcaagccgtgtgagccgtcgcagccgtggcatcgttgaggagtgctgtttccgcagctgtgacctggccctcctggagacgtactgtgctacccccgccaagtccgagagggacgtgtcgacccctccgaccgtgcttccggacaacttccccagataccccgtgggcaagttcttccaatatgacacctggaagcagtccacccagcgcctgcgcaggggcctgcctgccctcctgcgtgcccgccggggtcacgtgctcgccaaggagctcgaggcgttcagggaggccaaacgtcaccgtcccctgattgctctacccacccaagaccccgcccacgggggcgcccccccagagatggccagcaatcggaag

SEQ ID NO: 3 - protein, 236 aa, Homo sapiens
MVSPDPQIIVVAPETELASMQVQRTEDGVTIIQIFWVGRKGELLRRTPVSSAMQTPMGIPMGKSMLVLLTFLAFASCCIAAYRPSETLCGGELVDTLQFVCGDRGFYFSRPASRVSRRSRGIVEECCFRSCDLALLETYCATPAKSERDVSTPPTVLPDNFPRYPVGKFFQYDTWKQSTQRLRRGLPALLRARRGHVLAKELEAFREAKRHRPLIALPTQDPAHGGAPPEMASNRK

SEQ ID NO: 4 - DNA, 540 bp, Homo sapiens; identical to SEQ ID NO: 2

SEQ ID NO: 5 - protein, 57 aa, Homo sapiens
AYRPSETLCGGELVDTLQFVCGDRGFYFSRPASRVSRRSRGIVEECCFRSCDLALLE

SEQ ID NO: 6 - DNA, 171 bp, Homo sapiens
gcttaccgccccagtgagaccctgtgcggcggggagctggtggacaccctccagttcgtctgtggggaccgcggcttctacttcagcaggcccgcaagccgtgtgagccgtcgcagccgtggcatcgttgaggagtgctgtttccgcagctgtgacctggccctcctggag

SEQ ID NO: 7 - protein, 191 aa, Mus musculus
MGGSVAGFQVPMGIPVGKSMLVLLISLAFALCCIAAYGPGETLCGGELVDTLQFVCSDRGFYFSRPSSRANRRSRGIVEECCFRSCDLALLETYCATPAKSERDVSTSQAVLPDDFPRYPVGKFFQYDTWRQSAGRLRRGLPALLRARRGRMLAKELKEFREAKRHRPLIVLPPKDPAHGGASSEMSSNHQ

SEQ ID NO: 8 - DNA, 573 bp, Mus musculus
atgggcggcagcgtcgccggcttccaggtaccaatggggatcccagtggggaagtcgatgttggtgcttctcatctctttggccttcgccttgtgctgcatcgctgcttacggccccggagagactctgtgcggaggggagcttgttgacacgcttcagtttgtctgttcggaccgcggcttctacttcagcaggccttcaagccgtgccaaccgtcgcagccgtggcatcgtggaagagtgctgcttccgcagctgcgacctggccctcctggagacatactgtgccacccccgccaagtccgagagggacgtgtctacctctcaggccgtacttccggacgacttccccagataccccgtgggcaagttcttccaatatgacacctggagacagtccgcgggacgcctgcgcagaggcctgcctgccctcctgcgtgcccgccggggtcgcatgcttgccaaagagctcaaagagttcagagaggccaaacgtcatcgtcccctgatcgtgttaccacccaaagaccccgcccacgggggagcctcttcggagatgtccagcaaccatcag

SEQ ID NO: 9 - protein, 180 aa, Mus musculus
MGIPVGKSMLVLLISLAFALCCIAAYGPGETLCGGELVDTLQFVCSDRGFYFSRPSSRANRRSRGIVEECCFRSCDLALLETYCATPAKSERDVSTSQAVLPDDFPRYPVGKFFQYDTWRQSAGRLRRGLPALLRARRGRMLAKELKEFREAKRHRPLIVLPPKDPAHGGASSEMSSNHQ

SEQ ID NO: 10 - DNA, 540 bp, Mus musculus
atggggatcccagtggggaagtcgatgttggtgcttctcatctctttggccttcgccttgtgctgcatcgctgcttacggccccggagagactctgtgcggaggggagcttgttgacacgcttcagtttgtctgttcggaccgcggcttctacttcagcaggccttcaagccgtgccaaccgtcgcagccgtggcatcgtggaagagtgctgcttccgcagctgcgacctggccctcctggagacatactgtgccacccccgccaagtccgagagggacgtgtctacctctcaggccgtacttccggacgacttccccagataccccgtgggcaagttcttccaatatgacacctggagacagtccgcgggacgcctgcgcagaggcctgcctgccctcctgcgtgcccgccggggtcgcatgcttgccaaagagctcaaagagttcagagaggccaaacgtcatcgtcccctgatcgtgttaccacccaaagaccccgcccacgggggagcctcttcggagatgtccagcaaccatcag

SEQ ID NO: 11 - protein, 67 aa, Mus musculus
AYGPGETLCGGELVDTLQFVCSDRGFYFSRPSSRANRRSRGIVEECCFRSCDLALLETYCATPAKSE

SEQ ID NO: 12 - DNA, 201 bp, Mus musculus
gcttacggccccggagagactctgtgcggaggggagcttgttgacacgcttcagtttgtctgttcggaccgcggcttctacttcagcaggccttcaagccgtgccaaccgtcgcagccgtggcatcgtggaagagtgctgcttccgcagctgcgacctggccctcctggagacatactgtgccacccccgccaagtccgag

SEQ ID NO: 13 - protein, 67 aa, Homo sapiens
AYRPSETLCGGELVDTLQFVCGDRGFYFSRPASRVSRRSRGIVEECCFRSCDLALLETYCATPAKSE

SEQ ID NO: 14 - DNA, 201 bp, Homo sapiens
gcttaccgccccagtgagaccctgtgcggcggggagctggtggacaccctccagttcgtctgtggggaccgcggcttctacttcagcaggcccgcaagccgtgtgagccgtcgcagccgtggcatcgttgaggagtgctgtttccgcagctgtgacctggccctcctggagacgtactgtgctacccccgccaagtccgag

(19) United States
(12) Patent Application Publication
(10) Pub. No.: US 2023/0145514 A1
(43) Pub. Date: May 11, 2023

(54) USE OF IGF-2 FOR TREATMENT OF EPILEPTIC SEIZURES

(71) Applicant: New York University, New York, NY (US)

(72) Inventors: Cristina Maria ALBERINI, New York, NY (US); Emmanuel CRUZ, New York, NY (US)

(21) Appl. No.: 17/996,862

(22) PCT Filed: Apr. 23, 2021
(86) PCT No.: PCT/US2021/028947

§ 371 Date: Oct. 21, 2022

Related U.S. Application Data
(60) Provisional application No. 63/015,181, filed on Apr. 24, 2020.

(51) Int. Cl. A61K 38/30; A61P 25/08

(52) U.S. Cl. CPC A61K 38/30; A61P 25/08

(57) ABSTRACT

Provided are methods for treatment of seizures associated with neurodevelopmental disorders, such as Angelman Syndrome and autism comprising administering to an individual a composition comprising IGF-2.

### CROSS REFERENCE TO RELATED APPLICATIONS

This application claims priority to U.S. provisional patent application No. 63/015,181 filed on Apr. 24, 2020, the contents of which are incorporated by reference in their entirety herein.

### STATEMENT REGARDING FEDERALLY SPONSORED RESEARCH OR DEVELOPMENT

This invention was made with government support under grant number NM065635 awarded by the National Institutes of Health. The government has certain rights in the invention.

### INCORPORATION BY REFERENCE OF SEQUENCE LISTING

The present application is being filed with a Sequence Listing in electronic format. The Sequence Listing is provided as a file entitled RTTP-006-01WO_SeqList.txt, created on Apr. 6, 2021 and is 14 kilobytes in size. The information in electronic format of the Sequence Listing is incorporated by reference in its entirety.

### BACKGROUND

Epileptic seizures are often non-responsive to many prescribed medications. Epileptic seizures are caused by many neurodevelopmental and neurological disorders. One such disorder, Angelman Syndrome (AS), is a neurological disorder that occurs in one in about 20,000 live births. Characteristics or symptoms of AS include developmental delay, lack of speech, walking and balance disorders, and seizures. Some of the characteristics of Angelman Syndrome overlap with autism spectrum disorders, although the two conditions have their own unique characteristics. Currently, there is no known cure for AS or autism, or seizures caused by these or other disorders, or no viable therapeutic approaches to ameliorate the various symptoms associated with these indications.

### SUMMARY

The present disclosure provides methods for treatment of seizures, including seizures associated with neurodevelopmental disorders, such as Angelman Syndrome (AS) and autism. The methods comprise administering to a subject in need of treatment, a composition comprising an insulin-like growth factor 2 (IGF-2 or IGF-II) or derivatives or analogs thereof. For example, the compositions may comprise, or consist essentially of, a therapeutically effect amount of IGF-2 or derivatives thereof.

In one aspect, this disclosure pertains to a method of preventing, reversing and/or treating seizures in a subject comprising administering to the subject a composition comprising a therapeutically effective amount of IGF-2, or a functional modification or derivative thereof.

In one aspect, this disclosure pertains to a method of preventing, reversing and/or treating seizures in a subject comprising administering to the subject a composition consisting essentially of a therapeutically effective amount of IGF-2, or a functional modification or derivative thereof.

In one aspect, this disclosure pertains to a method of treating seizures in a subject comprising administering to the subject a composition comprising a therapeutically effective amount of IGF-2, or a functional modification or derivative thereof.

In one aspect, this disclosure pertains to a method of treating seizures in a subject comprising administering to the subject a composition consisting essentially of a therapeutically effective amount of IGF-2, or a functional modification or derivative thereof.

In one embodiment, the subject has been diagnosed with a neurodevelopmental or neurological disorder.

In one embodiment, the neurodevelopmental or neurological disorder comprises autism spectrum disorder (ASD), Angelman Syndrome (AS), cerebral palsy, Ohtahara Syndrome, benign familial neonatal seizures, West Syndrome, Dravet Syndrome, Rett Syndrome, Tuberous sclerosis, Sturge-Weber Syndrome, Landau-Kleffner Syndrome, Lennox-Gastaut Syndrome, Rasmussen Syndrome, Gelastic Epilepsy, Benign Rolandic Epilepsy, Panayiotopoulos syndrome, Gastaut-type Syndrome, childhood absence epilepsy, or juvenile myoclonic epilepsy.

In one embodiment, the neurodevelopmental or neurological disorder comprises autism spectrum disorder or Angelman Syndrome.

In one embodiment, the subject has a seizure-inducing disease or disorder.

In one embodiment, the seizure-inducing disease or disorder comprises an autoimmune disorder, a cerebral edema, cerebral ischema or hypoxia, head trauma, a central nervous system infection, an intracranial lesion, hyperpyrexia, a metabolic disorder, or a neurocutaneous disorder.

In one embodiment, the subject has been exposed to a seizure-inducing drug or toxin.

In one embodiment, the subject has been diagnosed with a congenital abnormality that causes seizures.

In one embodiment, the subject has been diagnosed with epilepsy.

In one embodiment, the seizures comprise focal seizures or generalized seizures.

In one embodiment, the focal seizures comprise focal seizures without loss of consciousness or focal seizures with impaired awareness.

In one embodiment, the generalized seizures comprise absence seizures, tonic seizures, atonic seizures, clonic seizures, myoclonic seizures, or tonic-clonic seizures.

In one embodiment, the seizures are audiogenic seizures.

In one embodiment, the IGF-2 comprises a sequence of SEQ ID NO: 1 or 3, or a sequence having at least 95% identity thereto.

In one embodiment, the IGF-2 comprises a sequence of SEQ ID NO: 11 or 13, or a sequence having at least 95% identity thereto.

In one embodiment, the treatment comprises alleviating one or more symptoms of epilepsy.

In one embodiment, the treatment reduces the severity, duration or frequency of a seizure.

In one embodiment, the IGF-2 is administered to the subject in an amount in the range of 1 to 500 μg/kg of the subject's body weight.

In one embodiment, the IGF-2 is the only agent in the composition that is capable of binding to IGF-2 receptor.

In one embodiment, the composition comprises one or more pharmaceutically acceptable carrier, diluent, or excipient.

In one embodiment, the composition is administered to the subject orally, intranasally, topically, anally, parenterally, intramuscularly, intraperitoneally, intravenously, intracerebral, ocularly, optically, intracerebrally, intraspinally intrathecally, subcutaneously.

In one aspect, the application pertains to the use of a composition comprising a therapeutically effective amount of IGF-2, or a functional modification or derivative thereof, in the manufacture of a medicament for the preventing, reversing and/or treating of seizures in a subject.

In one aspect, the application pertains to the use of a composition comprising a therapeutically effective amount of IGF-2, or a functional modification or derivative thereof, in the manufacture of a medicament for the treating of seizures in a subject.

In one aspect, the application pertains to a composition comprising a therapeutically effective amount of IGF-2, or a functional modification or derivative thereof, for use in the prevention, reversion, or treatment of seizures in a subject.

In one aspect, the application pertains to a composition comprising a therapeutically effective amount of IGF-2, or a functional modification or derivative thereof, for use in the treatment of seizures in a subject.

In one embodiment, the subject has been diagnosed with a neurodevelopmental or neurological disorder.

In one embodiment, the neurodevelopmental or neurological disorder is autism spectrum disorder (ASD), Angelman Syndrome (AS), cerebral palsy, Ohtahara Syndrome, benign familial neonatal seizures, West Syndrome, Dravet Syndrome, Rett Syndrome, Tuberous sclerosis, Sturge-Weber Syndrome, Landau-Kleffner Syndrome, Lennox-Gastaut Syndrome, Rasmussen Syndrome, Gelastic Epilepsy, Benign Rolandic Epilepsy, Panayiotopoulos syndrome, Gastaut-type Syndrome, childhood absence epilepsy, or juvenile myoclonic epilepsy.

In one embodiment, the IGF-2 comprises a sequence of SEQ ID NO: 1 or 3, or a sequence having at least 95% identity thereto.

In one embodiment, the IGF-2 comprises a sequence of SEQ ID NO: 11 or 13, or a sequence having at least 95% identity thereto.

### BRIEF DESCRIPTION OF THE DRAWINGS

FIGS. 1A-D shows that IGF-2, but not M6P, attenuates audiogenic seizures of Ube3a^{m-/p+} mice. A subcutaneous (s.c.) injection of IGF2, M6P, or vehicle was administered 20 minutes before audiogenic seizure induction. (FIG. 1A) Percentage of Ube3am−/p+ mice injected with vehicle or IGF2 exhibiting wild-running and/or tonic-clonic seizures following an audiogenic stimulus. Chi-square test, *p<0.05. (FIG. 1B) Percent of Ube3a^{m-/p+} mice injected with vehicle or M6P exhibiting wild-running and/or tonic-clonic seizures following an audiogenic stimulus. (FIG. 1C) Severity score of audiogenic seizures; 0=no seizure response; 1=wild-running; 2=tonic-clonic seizure. Kruskal-Wallis test with Dunn's multiple comparisons, *p<0.05. (FIG. 1D) Graph summarizing the percentage of wild running, tonic-clonic or protection from both wild running and tonic-clonic seizure in Ube3a^{m-/p+} mice with vehicle, IGF2, or M6P treatment. N=8-11/group.

FIGS. 2A-2C show Table 1. A subcutaneous (s.c.) IGF-2 or M6P injection does not produce adverse effects in either WT or Ube3a^{m-/p+} mice. An observational battery of general physical characteristics and motor and sensory responses of WT and Ube3a^{m-/p+} (AS) mice injected with IGF-2, M6P or Vehicle (Veh) tested 30 min (FIG. 2A), 24 hours (h) (FIG. 2B) and 7 days (d) after injection (FIG. 2C) are shown.

### DETAILED DESCRIPTION

This disclosure provides methods for the treatment, prevention, or amelioration of seizures, such as seizures associated with neurodegenerative disorders, such as Angelman Syndrome and autism spectrum disorder. The methods relate to administration of IGF-2 and/or IGF-2 modifications to an individual in need of treatment.

### Definitions

The term “treatment” as used herein refers to reduction or delay in one or more symptoms or features associated with the presence of the particular condition being treated, e.g., Angelman syndrome or autistic disorders, and in particular with seizures associated with AS or autistic disorders. Treatment does not mean complete cure. For example, treatment of AS in the present disclosure means reducing, reversing or preventing seizures or associated symptoms.

“Treating” or “treatment” of a state, disorder or condition includes: preventing or delaying the appearance or slowing down the progression of clinical or sub-clinical symptoms of the state, disorder or condition developing in a mammal that may be afflicted with or predisposed to the state, disorder or condition but does not yet experience or display clinical or subclinical symptoms of the state, disorder or condition; or inhibiting the state, disorder or condition, e.g., arresting, reducing or delaying the development of the disease or a relapse thereof (in case of maintenance treatment) or at least one clinical or sub-clinical symptom thereof; or relieving the disease, e.g., causing regression of the state, disorder or condition or at least one of its clinical or sub-clinical symptoms. The benefit to a subject to be treated is either statistically significant or at least perceptible to the subject or to the person administering the treatment (e.g., a physician). For example, in treating seizures, the benefit may be to decrease seizure frequency, severity or duration.

The term “therapeutically effective amount” as used herein is the amount sufficient to achieve, in a single or multiple doses, the intended purpose of treatment. For example, an effective amount to treat seizures is an amount sufficient to alleviate seizures such as those associated with AS or autism. The exact amount desired or required will vary depending on the mode of administration, patient specifics and the like. Appropriate effective amounts can be determined by one of ordinary skill in the art (such as a clinician) with the benefit of the present disclosure.

“Homology” refers to the percent identity between two polynucleotide or two polypeptide molecules. Two nucleic acid, or two polypeptide sequences are “substantially homologous” or “substantially identical” to each other when the sequences exhibit at least about 50% sequence identity, preferably at least about 75% sequence identity, more preferably at least about 80%-85% sequence identity, more preferably at least about 90% sequence identity, and most preferably at least about 95%-98% sequence identity over a defined length of the molecules.

In general, “identity” refers to an exact nucleotide to nucleotide or amino acid to amino acid correspondence of two polynucleotides or polypeptide sequences, respectively. Percent identity can be determined by a direct comparison of the sequence information between two molecules by aligning the sequences, counting the exact number of matches between the two aligned sequences, dividing by the length of the shorter sequence, and multiplying the result by 100. Readily available computer programs can be used to aid in the analysis. Programs for determining nucleotide sequence identity are available in the Wisconsin Sequence Analysis Package, Version 8 (available from Genetics Computer Group, Madison, Wis.) for example, the BESTFIT, FASTA and GAP programs, which also rely on the Smith and Waterman algorithm. Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non redundant, GenBank+EMBL+DDBJ+PDB+GenBank CDS translations+Swiss protein+Spupdate+PIR. Details of these programs are readily available.

“Recombinant” as used herein to describe a nucleic acid molecule means a polynucleotide of genomic, cDNA, viral, semisynthetic, or synthetic origin which, by virtue of its origin or manipulation, is not associated with all or a portion of the polynucleotide with which it is associated in nature. The term “recombinant” as used with respect to a protein or polypeptide means a polypeptide produced by expression of a recombinant polynucleotide. In general, the gene of interest is cloned and then expressed in transformed organisms, as described further below. The host organism expresses the foreign gene to produce the protein under expression conditions.

By an “isolated” polypeptide or a variant, or derivative thereof is intended a polypeptide that is not in its natural milieu. No particular level of purification is required. For example, an isolated polypeptide can be removed from its native or natural environment. Recombinantly produced polypeptides and proteins expressed in host cells are considered isolated for the purpose of the invention, as are native or recombinant polypeptides which have been separated, fractionated, or partially or substantially purified by any suitable technique.

Where a range of values is provided in this disclosure, it should be understood that each intervening value, to the tenth of the unit of the lower limit between the upper and lower limit of that range, and any other intervening value in that stated range is encompassed within the invention, unless clearly indicated otherwise. The upper and lower limits of these smaller ranges may independently be included in the smaller ranges encompassed within the disclosure.

As used in this disclosure, the singular forms include the plural forms and vice versa unless the context clearly indicates otherwise.

In an aspect, this disclosure provides a method of treatment of seizures comprising administering to a subject in need of treatment, a composition comprising IGF-2 or a modification thereof. In an embodiment, this disclosure provides a method of treatment of seizures associated with neurodevelopmental disorder such as Angelman syndrome or autism by administering to a subject in need of treatment, a composition comprising IGF-2 or a modification thereof.

In this disclosure, the terms “individual” and “subject” may be used interchangeably. The subject may be any animal subject, such as a human, a laboratory animal, or any other animal. Derivatives or analogs such as a modified IGF-2 or IGF-2 with amino acid substitutions such as human Leu 27 (Armitaj et al., Neuroscience, 2010 Oct. 27; 170(3):722-30) may also be used.

All publications, patents, and patent applications mentioned in this specification are herein incorporated by reference to the same extent as if each individual publication, patent, or patent application was specifically and individually indicated to be incorporated by reference.

### Insulin-Like Growth Factor 2 (IGF-2)

The disclosure provides methods of treating, preventing or reversing a seizure comprising administering a composition comprising IGF-2, or a functional modification or derivative thereof.

Insulin-like growth factor 2 (IGF-2, also called IGF2 and IGF-II) is a polypeptide with structural homology to insulin and, together with insulin-like growth factor 1 (IGF-1, also known as IGF, IGF1 and MGF), belongs to the IGF/IGFBP system. IGF-2 is still poorly characterized: in adult tissues, its expression remains relatively high in brain regions, including the hippocampus, but is also found in the hypothalamus, striatum, cortex and cerebellum and declines with aging. Numerous studies indicate that IGF-2 and IGF-1 play similar functions including promoting neuronal survival and protection against injury, however these effects are likely due to the activation of the IGF-1 receptor. In fact, IGF-2 has been found to be involved in promoting neuronal survival, proliferation and maturation, reduction of neuronal loss in adult brain following hypoxic-ischemic injury, protection of oligodendrocytes and hippocampal septal neurons, neurite outgrowth and direct sprouting of spared afferent into a de-afferent hippocampus. These effects may occur via IGF-1 receptor to which IGF-2 can bind with low affinity, hence the activation of the IGF-1 pathway. IGF-2 has shown distinct functions via activation of the IGF-2 receptor.

IGF-2 has also been found to be involved in synaptic plasticity and memory. Administration of recombinant IGF-2, either intracerebrally or systemically (subcutaneously, s.c.), significantly enhances memory retention and persistence in healthy mice and rats. In BTBR T+ Itpr3tf/J (BTBR) mice, a model that reproduces most of the core behavioral phenotypes of autism spectrum disorder (ASD), a s.c. injection of IGF-2 reversed cognitive and social impairments, as well as repetitive behaviors, and significantly ameliorates the underlying deficits of the AMPK-mTOR-S6K pathway and increased protein synthesis. Sucutaneous injections of IGF-2 also reverse cognitive impairments, motor problems and repetitive behaviors in a mouse model of Angelman syndrome (Ube3a^{m-/p+}) IGF-2 injected into the hippocampus reverses aging-related memory loss in rats. Furthermore, IGF-2 hippocampal overexpression or intraventricular injection rescues memory impairments, amyloid plaque load, and cholinergic dysfunctions in multiple mouse models of Alzheimer's disease. Finally, IGF-2 is neuro-protective in models of neuronal oxidative damage. Where assessed, these positive effects of IGF-2 were found to be mediated by its high-affinity receptor, the IGF-2 receptor (IGF-2R), also known as cation-independent mannose-6-phosphate receptor (CIM6PR), and not by the IGF-1 receptor to which IGF-2 can bind with lower affinity.

IGF-2 binds with high affinity to the insulin like growth factor 2 receptor (CIM6P/IGF-2R). CIM6P/IGF-2R is a single transmembrane protein that belongs to the IGF/insulin system but acts distinctively from the other receptors of this system, (IGF-1R and insulin receptors) by regulating endosomal protein trafficking and lysosomal targeting. In fact, while IGF-1 receptor and Insulin receptors are receptor tyrosine kinase linked to the activation of the classical growth pathways involved in cell growth, proliferation, and survival, IGF-2R binds both molecules bearing M6P and IGF-2 and its main known function is to traffic lysosomal enzymes and lead IGF-2 to lysosomal degradation. Recent studies showed that IGF-2R expressed in the brain, and particularly the hippocampus, is required for memory formation acting via the control of de novo protein synthesis induced by learning (Yu et al. 2020; 9:e54781. doi: 10.7554/eLife.54781; 2020).

Data indicate that the beneficial effects of IGF-2 administration occur through CIM6P/IGF-2R. The mechanisms of action underlying the therapeutic effects of CIM6P/IGF-2R ligands are currently unclear. However, in healthy rodent brains, data show that IGF-2R in the hippocampus is required for memory formation by controlling learning-induced de novo protein synthesis, which is coupled to autophagy. IGF-2 administration promotes autophagic flux, measured by a reporter of autophagy (LC3B) that allows the detection of the rate of progressive acidification and degradation activity. Without wishing to be bound by theory, it is thought that one mechanism by which IGF-2 treats seizures is through CIM6P/IGF-2R regulated activity- and experience-dependent homeostasis of protein metabolism, also known as proteostasis. Alternatively, or in addition, CIM6P/IGF-2R is thought to be essential for endosomal trafficking and lysosomal targeting. Given its involvement in targeting lysosomal enzymes and functions, CIM6P/IGF-2R is thought to be engaged in regulating protein metabolism, and in particular protein degradation via lysosomal targeting and its crosstalk with proteasome-mediated protein degradation, which could mediate that therapeutic effects of increased CIM6P/IGF-2R signaling.

A representative full length human IGF-2 isoform 1 is described at NCBI accession number NP_001278791.1, and comprises a sequence of: 1 MGIPMGKSML VLLTFLAFAS CCIAAYRPSE TLCGGELVDT LQFVCGDRGF YFSRPASRVS 61 RRSRGIVEEC CFRSCDLALL ETYCATPAKS ERDVSTPPTV LPDNFPRYPV GKFFQYDTWK 121 QSTQRLRRGL PALLRARRGH VLAKELEAFR EAKRHRPLIA LPTQDPAHGG APPEMASNRK (SEQ ID NO: 1). In some embodiments, IGF-2 comprises a sequence of SEQ ID NO: 1, or a sequence having at least 90%, at least 95%, at least 96%, at least 97%, at least 98% or at least 99% identity thereto. In some embodiments, IGF-2 comprises a sequence of SEQ ID NO: 1. In some embodiments, IGF-2 comprises a functional fragment of SEQ ID NO: 1.

In some embodiments, IGF-2 isoform 1 is encoded by a polynucleotide comprising a sequence of: 1 atgggaatcc caatggggaa gtcgatgctg gtgcttctca ccttcttggc cttcgcctcg 61 tgctgcattg ctgcttaccg ccccagtgag accctgtgcg gcggggagct ggtggacacc 121 ctccagttcg tctgtgggga ccgcggcttc tacttcagca ggcccgcaag ccgtgtgagc 181 cgtcgcagcc gtggcatcgt tgaggagtgc tgtttccgca gctgtgacct ggccctcctg 241 gagacgtact gtgctacccc cgccaagtcc gagagggacg tgtcgacccc tccgaccgtg 301 cttccggaca acttccccag ataccccgtg ggcaagttct tccaatatga cacctggaag 361 cagtccaccc agcgcctgcg caggggcctg cctgccctcc tgcgtgcccg ccggggtcac 421 gtgctcgcca aggagctcga ggcgttcagg gaggccaaac gtcaccgtcc cctgattgct 481 ctacccaccc aagaccccgc ccacgggggc gcccccccag agatggccag caatcggaag (SEQ ID NO: 2). In some embodiments, IGF-2 isoform 1 is encoded by a polynucleotide comprising as SEQ ID NO: 2, or a sequence having at least 90%, at least 95%, at least 96%, at least 97%, at least 98% or at least 99% identity thereto. In some embodiments, IGF-2 isoform 1 is encoded by a polynucleotide comprising as SEQ ID NO: 2.

A representative full length human IGF-2 isoform 2 is described at NCBI accession number NP_001121070.1, and comprises a sequence of:

| (SEQ ID NO: 3) | |
|---|---|
| 1 | MVSPDPQIIV VAPETELASM QVQRTEDGVT IIQIFWVGRK GELLRRTPVS SAMQTPMGIP |
| 61 | MGKSMLVLLT FLAFASCCIA AYRPSETLCG GELVDTLQFV CGDRGFYFSR PASRVSRRSR |
| 121 | GIVEECCFRS CDLALLETYC ATPAKSERDV STPPTVLPDN FPRYPVGKFF QYDTWKQSTQ |
| 181 | RLRRGLPALL RARRGHVLAK ELEAFREAKR HRPLIALPTQ DPAHGGAPPE MASNRK. |

In some embodiments, IGF-2 comprises a sequence of SEQ ID NO: 3, or a sequence having at least 90%, at least 95%, at least 96%, at least 97%, at least 98% or at least 99% identity thereto. In some embodiments, IGF-2 comprises a sequence of SEQ ID NO: 3. In some embodiments, IGF-2 comprises a functional fragment of SEQ ID NO: 3.

In some embodiments, IGF-2 isoform 2 is encoded by a polynucleotide comprising a sequence of: 1 atgggaatcc caatggggaa gtcgatgctg gtgcttctca ccttcttggc cttcgcctcg 61 tgctgcattg ctgcttaccg ccccagtgag accctgtgcg gcggggagct ggtggacacc 121 ctccagttcg tctgtgggga ccgcggcttc tacttcagca ggcccgcaag ccgtgtgagc 181 cgtcgcagcc gtggcatcgt tgaggagtgc tgtttccgca gctgtgacct ggccctcctg 241 gagacgtact gtgctacccc cgccaagtcc gagagggacg tgtcgacccc tccgaccgtg 301 cttccggaca acttccccag ataccccgtg ggcaagttct tccaatatga cacctggaag 361 cagtccaccc agcgcctgcg caggggcctg cctgccctcc tgcgtgcccg ccggggtcac 421 gtgctcgcca aggagctcga ggcgttcagg gaggccaaac gtcaccgtcc cctgattgct 481 ctacccaccc aagaccccgc ccacgggggc gcccccccag agatggccag caatcggaag (SEQ ID NO: 4). In some embodiments, IGF-2 isoform 2 is encoded by a polynucleotide comprising as SEQ ID NO: 4, or a sequence having at least 90%, at least 95%, at least 96%, at least 97%, at least 98% or at least 99% identity thereto.

In some embodiments, IGF-2 of the instant disclosure comprises a sequence of AYRPSETLCGGELVDTLQFVCGDRGFYFSRPASRVSRRSRGIVEECCFRSCDLALLETYCA TPAKSE (SEQ ID NO: 13), or a sequence having at least 90%, at least 95%, at least 96%, at least 97%, at least 98% or at least 99% identity thereto. In some embodiments, IGF-2 comprises or consists essentially of SEQ ID NO: 13. SEQ ID NO: 13 is a fragment of full length human IGF-2, corresponding to Alanine 25 through Glutamate 91 of SEQ ID NO: 1.

In some embodiments, IGF-2 is encoded by a sequence of: 1 gcttaccgcc ccagtgagac cctgtgcggc ggggagctgg tggacaccct ccagttcgtc 61 tgtggggacc gcggcttcta cttcagcagg cccgcaagcc gtgtgagccg tcgcagccgt 121 ggcatcgttg aggagtgctg tttccgcagc tgtgacctgg ccctcctgga gacgtactgt 181 gctacccccg ccaagtccga g (SEQ ID NO: 14). In some embodiments, IGF-2 comprises a sequence of SEQ ID NO: 14, or a sequence having at least 90%, at least 95%, at least 96%, at least 97%, at least 98% or at least 99% identity thereto. In some embodiments, IGF-2 comprises a sequence of SEQ ID NO: 14.

In some embodiments, IGF-2 of the instant disclosure comprises a sequence of AYRPSETLCGGELVDTLQFVCGDRGFYFSRPASRVSRRSRGIVEECCFRSCDLALLE (SEQ ID NO: 5), or a sequence having at least 90%, at least 95%, at least 96%, at least 97%, at least 98% or at least 99% identity thereto. In some embodiments, IGF-2 comprises or consists essentially of SEQ ID NO: 5. SEQ ID NO: 5 is a fragment of full length human IGF-2, corresponding to Alanine 25 through Glutamate 81 of SEQ ID NO: 1. Other IGF-2 peptide fragments can be generated from SEQ ID NO: 1 or SEQ ID NO: 3, and are envisaged as within the scope of the instant disclosure. The person of ordinary skill will appreciate that a peptide fragment of IGF-2 can differ from SEQ ID NO: 1 or 3 at the N or C terminus by several amino acids, e.g., 1, 2, 3, 4, 5, 6, 7, 8, 9, 10, or more amino acids, and still retain IGF-2 function.

In some embodiments, IGF-2 is encoded by a sequence of:

| (SEQ ID NO: 6) | |
|---|---|
| 1 | gcttaccgcc ccagtgagac cctgtgcggc ggggagctgg tggacaccct ccagttcgtc |
| 61 | tgtggggacc gcggcttcta cttcagcagg cccgcaagcc gtgtgagccg tcgcagccgt |
| 121 | ggcatcgttg aggagtgctg tttccgcagc tgtgacctgg ccctcctgga g. |

In some embodiments, IGF-2 isoform 2 is encoded by a polynucleotide comprising as SEQ ID NO: 6, or a sequence having at least 90%, at least 95%, at least 96%, at least 97%, at least 98% or at least 99% identity thereto.

A representative full length mouse IGF-2 isoform 1 precursor is described at NCBI accession number NP_034644.2, and comprises a sequence of: 1 MGGSVAGFQV PMGIPVGKSM LVLLISLAFA LCCIAAYGPG ETLCGGELVD TLQFVCSDRG 61 FYFSRPSSRA NRRSRGIVEE CCFRSCDLAL LETYCATPAK SERDVSTSQA VLPDDFPRYP 121 VGKFFQYDTW RQSAGRLRRG LPALLRARRG RMLAKELKEF REAKRHRPLI VLPPKDPAHG 181 GASSEMSSNH Q (SEQ ID NO: 7). In some embodiments, IGF-2 comprises a sequence of SEQ ID NO: 7, or a sequence having at least 90%, at least 95%, at least 96%, at least 97%, at least 98% or at least 99% identity thereto. In some embodiments, IGF-2 comprises a sequence of SEQ ID NO: 7. in some embodiments, IGF-2 comprises a functional fragment of SEQ ID NO: 7.

In some embodiments, mouse IGF-2 isoform 1 is encoded by a polynucleotide comprising a sequence of: 1 atgggcggca gcgtcgccgg cttccaggta ccaatgggga tcccagtggg gaagtcgatg 61 ttggtgcttc tcatctcttt ggccttcgcc ttgtgctgca tcgctgctta cggccccgga 121 gagactctgt gcggagggga gcttgttgac acgcttcagt ttgtctgttc ggaccgcggc 181 ttctacttca gcaggccttc aagccgtgcc aaccgtcgca gccgtggcat cgtggaagag 241 tgctgcttcc gcagctgcga cctggccctc ctggagacat actgtgccac ccccgccaag 301 tccgagaggg acgtgtctac ctctcaggcc gtacttccgg acgacttccc cagatacccc 361 gtgggcaagt tcttccaata tgacacctgg agacagtccg cgggacgcct gcgcagaggc 421 ctgcctgccc tcctgcgtgc ccgccggggt cgcatgcttg ccaaagagct caaagagttc 481 agagaggcca aacgtcatcg tcccctgatc gtgttaccac ccaaagaccc cgcccacggg 541 ggagcctctt cggagatgtc cagcaaccat cag (SEQ ID NO: 8). In some embodiments, IGF-2 isoform 1 is encoded by a polynucleotide comprising as SEQ ID NO: 8, or a sequence having at least 90%, at least 95%, at least 96%, at least 97%, at least 98% or at least 99% identity thereto.

A representative full length mouse IGF-2 isoform 2 preprotein is described at NCBI accession number NP_001116208.1, and comprises a sequence of: 1 MGIPVGKSML VLLISLAFAL CCIAAYGPGE TLCGGELVDT LQFVCSDRGF YFSRPSSRAN 61 RRSRGIVEEC CFRSCDLALL ETYCATPAKS ERDVSTSQAV LPDDFPRYPV GKFFQYDTWR 121 QSAGRLRRGL PALLRARRGR MLAKELKEFR EAKRHRPLIV LPPKDPAHGG ASSEMSSNHQ (SEQ ID NO: 9). In some embodiments, IGF-2 comprises a sequence of SEQ ID NO: 9, or a sequence having at least 90%, at least 95%, at least 96%, at least 97%, at least 98% or at least 99% identity thereto. In some embodiments, IGF-2 comprises a sequence of SEQ ID NO: 9. In some embodiments, IGF-2 comprises a functional fragment of SEQ ID NO: 9.

In some embodiments, mouse IGF-2 isoform 2 is encoded by a polynucleotide comprising a sequence of: 1 atggggatcc cagtggggaa gtcgatgttg gtgcttctca tctctttggc cttcgccttg 61 tgctgcatcg ctgcttacgg ccccggagag actctgtgcg gaggggagct tgttgacacg 121 cttcagtttg tctgttcgga ccgcggcttc tacttcagca ggccttcaag ccgtgccaac 181 cgtcgcagcc gtggcatcgt ggaagagtgc tgcttccgca gctgcgacct ggccctcctg 241 gagacatact gtgccacccc cgccaagtcc gagagggacg tgtctacctc tcaggccgta 301 cttccggacg acttccccag ataccccgtg ggcaagttct tccaatatga cacctggaga 361 cagtccgcgg gacgcctgcg cagaggcctg cctgccctcc tgcgtgcccg ccggggtcgc 421 atgcttgcca aagagctcaa agagttcaga gaggccaaac gtcatcgtcc cctgatcgtg 481 ttaccaccca aagaccccgc ccacggggga gcctcttcgg agatgtccag caaccatcag (SEQ ID NO: 10). In some embodiments, IGF-2 isoform 2 is encoded by a polynucleotide comprising as SEQ ID NO: 10, or a sequence having at least 90%, at least 95%, at least 96%, at least 97%, at least 98% or at least 99% identity thereto.

In some embodiments, IGF-2 of the instant disclosure comprises a fragment of full length IGF-2, for example a fragment of SEQ ID NO: 7 or SEQ ID NO: 9. In some embodiments, IGF-2 comprises a sequence of

AYGPGETLCGGELVDTLQFVCSDRGFYFSRPSSRANRRSRGIVEECCFRSCDLALL ETYCATPAKSE (SEQ ID NO: 11), or a sequence having at least 90%, at least 95%, at least 96%, at least 97%, at least 98% or at least 99% identity thereto. In some embodiments, IGF-2 comprises, or consists essentially of, SEQ ID NO: 11.

In some embodiments, IGF-2 is encoded by a polynucleotide comprising a sequence of: 1 gcttacggcc ccggagagac tctgtgcgga ggggagcttg ttgacacgct tcagtttgtc 61 tgttcggacc gcggcttcta cttcagcagg ccttcaagcc gtgccaaccg tcgcagccgt 121 ggcatcgtgg aagagtgctg cttccgcagc tgcgacctgg ccctcctgga gacatactgt 181 gccacccccg ccaagtccga g (SEQ ID NO: 12). In some embodiments, IGF-2 encoded by a polynucleotide comprising as SEQ ID NO: 12, or a sequence having at least 90%, at least 95%, at least 96%, at least 97%, at least 98% or at least 99% identity thereto.

Other IGF-2 peptide fragments can be generated from full length IGF-2 sequences such as SEQ ID NOS: 1, 3, 7 or 9, and are envisaged as within the scope of the instant disclosure. The person of ordinary skill will appreciate that a peptide fragment of IGF-2 can differ from full length IGF-2 peptides, for example SEQ ID NOS: 1, 3, 7 or 9, at the N or C terminus by several amino acids, e.g., 1, 2, 3, 4, 5, 6, 7, 8, 9, 10, or more amino acids, and still retain IGF-2 function.

As used herein, functional “derivatives” of IGF-2 include IGF-2 in which one or more amino acids have been substituted, deleted, or inserted, as long as such derivatives retain IGF-2 function. Functional derivatives of IGF-2 retain IGF-2R binding activity, and are capable of mediating signaling through IGF-2R. Persons of ordinary skill in the art will appreciate that amino acids of similar properties can be substituted for one another without loss of protein function, in some cases. For example, a non-polar amino acid such as glycine can be substituted for a similarly non-polar amino acid such as alanine, and similarly with polar, aromatic, positively charged or negatively charged amino acids. Further, by comparing IGF-2 sequences from different species, the person of ordinary skill in the art will understand which portions of IGF-2 are highly conserved, and which may be amenable to variation. Thus, functional derivatives of IGF-2 encompasse sequence variants of IGF-2. Fragments of IGF-2, such as SEQ ID NOS: 5 and 11, that retain IGF-2 function are also envisaged as within the scope of the instant disclosure. Functional derivatives of may also increase protein stability and bioavailability.

As used herein, “modifications” refers to any modification or alteration of a protein or peptide, e.g. IGF-2 of the disclosure. Proteins can be modified through the incorporation of non-natural amino acids, or through conjugation to other proteins or non-amino acid moieties. Modification can occur pre-translation, e.g. by incorporation of IGF-2 into a fusion protein, or post-translation. Modifications can enhance protein stability, and increase bioavailability. Exemplary post-translational modifications include, inter alia, glycosylation and phosphorylation. Proteins can also be modified by conjugation to carrier molecules that increase the half-life of the protein in vivo. For example, covalent attachment of a protein to a hydrophilic polymer such as poly (ethylene glycol), abbreviated PEG, can increase water solubility, bioavailability, serum half-life, therapeutic half-life, modulate biological activity, and extend the circulation time of many proteins. Modifications can increase stability, bioavailability, and improve formulations of recombinant proteins. For example, glycosylation, as well as covalent modifications, of recombinant insulin have been shown to increase stability.

Additional IGF-2 proteins and peptides, other than those described supra, are also within the scope of the instant disclosure. For example, the following IGF-2 proteins, and peptide fragments thereof, can be used in the methods, compounds and compositions of the disclosure. In some embodiments, the IGF-2 sequence corresponds to a sequence provided by Genbank Accession Nos.: P09535 (mouse), P01346 (rat), P01344 (human), P23695 (pig), P07456 (cow), P33717 (chicken), P51459 (horse), P10764 (sheep), AAI56000 (Xenopus), AAI70810 (Xenopus), AAI70812 (Xenopus), or a fragment thereof. IGF-2 sequences are known in the art, and depending on the subject to be treated, one of ordinary skill in the art will know which source (e.g., human, mouse, rat, etc.) of IGF-2 to use.

### Nucleic Acids and Vectors

The disclosure provides polynucleotides encoding the IGF-2 proteins described herein, and vectors comprising IGF-2 polynucleotides.

The IGF-2 polynucleotides of the present invention can be produced by recombinant DNA methods, well known to one of ordinary skill in the art. For example, an IGF-2 DNA sequence can be used in an expression construct to express the full length protein, or a fragment thereof.

The disclosure provides expression constructs comprising the IGF-2 polynucleotides described herein. An expression construct is a nucleic acid sequence comprising a target nucleic acid sequence (e.g., IGF-2) whose expression is desired, operatively associated with expression control sequence elements which provide for the proper transcription and translation of the target nucleic acid sequence(s) within the chosen host cells. Such sequence elements may include a promoter and a polyadenylation signal. Expression of suitable sequence elements will be known to persons of ordinary skill in the art. Exemplary promoters, include, inter alia, constitutive promoters such as the CMV, EF1A, and SV40 promoters. The expression construct may further comprise vector sequences. Vector sequences are any of several nucleic acid sequences established in the art which have utility in the recombinant DNA technologies of the invention to facilitate the cloning and propagation of the expression constructs including (but not limited to) plasmids, cosmids, phage vectors, viral vectors, and yeast artificial chromosomes.

Expression constructs of the present invention may comprise vector sequences that facilitate the cloning and propagation of the expression constructs which include a polynucleotide encoding IGF-2 or a derivative thereof. A large number of vectors, including plasmid and fungal vectors, have been described for replication and/or expression in a variety of eukaryotic and prokaryotic host cells. Standard vectors useful in the current invention are well known in the art and include (but are not limited to) plasmids, cosmids, phage vectors, viral vectors, and yeast artificial chromosomes. The vector sequences may contain a replication origin for propagation in E. coli; the SV40 origin of replication; an ampicillin, neomycin, or puromycin resistance gene for selection in host cells; and/or genes (e.g., dihydrofolate reductase gene) that amplify the dominant selectable marker plus the gene of interest.

Expression constructs of the present invention may comprise vector sequences that facilitate the production of IGF-2 protein. In general, the IGF-2 DNA sequence is expressed in or by a cell to form an IGF-2 expression product, such as a protein or peptide. The expression product itself (the resulting protein or peptide), may also be said to be “expressed” by the cell. An expression product can be characterized as intracellular, extracellular or secreted. Suitable cells and expression systems will be known to persons of skill in the art. The term “intracellular” means something that is inside a cell. The term “extracellular” means something that is outside a cell. A substance is “secreted” by a cell if it appears in significant measure outside the cell, from somewhere on or inside the cell.

Once the IGF-2 DNA sequence is inserted into a vector, or formulated into another expression construct, it is transformed into a host cell for expression as the respective protein or peptide. A host cell that receives and expresses introduced DNA or RNA has been transformed and is a transformant or a clone. The DNA or RNA introduced to a host cell can come from any source, including cells of the same genus or species as the host cell, or cells of a different genus or species.

IGF-2 polynucleotides may be readily isolated and sequenced using conventional procedures. In some embodiments a vector, for example an expression vector, comprising one or more of the polynucleotides of the invention is provided. Methods which are well known to those skilled in the art can be used to construct expression vectors containing the coding sequence of IGF-2 along with appropriate transcriptional/translational control signals. These methods include in vitro recombinant DNA techniques, synthetic techniques and in vivo recombination/genetic recombination. See, for example, the techniques described in Maniatis et al., MOLECULAR CLONING: A LABORATORY MANUAL, Cold Spring Harbor Laboratory, N.Y. (1989); and Ausubel et al., CURRENT PROTOCOLS IN MOLECULAR BIOLOGY, Greene Publishing Associates and Wiley Interscience, N.Y (1989).

In addition, a vector, polynucleotide, or nucleic acid of the invention may encode heterologous coding regions, either fused of unfused to a polynucleotide encoding the polypeptides of the invention, or variant or derivative thereof. Heterologous coding regions include without limitation specialized elements or motifs, such as a secretory signal peptide or a heterologous functional domain. An operable association is when a coding region for a gene product, e.g. a polypeptide, is associated with one or more regulatory sequences in such a way as to place expression of the gene product under the influence or control of the regulatory sequence(s). Two DNA fragments (such as a polypeptide coding region and a promoter associated therewith) are “operably associated” if induction of promoter function results in the transcription of mRNA encoding the desired gene product and if the nature of the linkage between the two DNA fragments does not interfere with the ability of the expression regulatory sequences to direct the expression of the gene product or interfere with the ability of the DNA template to be transcribed. Thus, a promoter region would be operably associated with a nucleic acid encoding a polypeptide if the promoter was capable of effecting transcription of that nucleic acid. The promoter may be a cell-specific promoter that directs substantial transcription of the DNA only in predetermined cells. Other transcription control elements, besides a promoter, for example enhancers, operators, repressors, and transcription termination signals, can be operably associated with the polynucleotide to direct cell-specific transcription. Suitable promoters and other transcription control regions are disclosed herein. A variety of transcription control regions are known to those skilled in the art. These include, without limitation, transcription control regions, which function in vertebrate cells, such as, but not limited to, promoter and enhancer segments from cytomegaloviruses (e.g. the immediate early promoter, in conjunction with intron-A), simian virus 40 (e.g. the early promoter), and retroviruses (such as, e.g. Rous sarcoma virus). Other transcription control regions include those derived from vertebrate genes such as actin, heat shock protein, bovine growth hormone and rabbit β-globin, as well as other sequences capable of controlling gene expression in eukaryotic cells. Additional suitable transcription control regions include tissue-specific promoters and enhancers as well as inducible promoters (e.g. promoters inducible tetracyclins). Similarly, a variety of translation control elements are known to those of ordinary skill in the art. These include, but are not limited to ribosome binding sites, translation initiation and termination codons, and elements derived from viral systems (particularly an internal ribosome entry site, or IRES, also referred to as a CITE sequence). The expression cassette may also include other features such as an origin of replication, and/or chromosome integration elements such as retroviral long terminal repeats (LTRs), or adeno-associated viral (AAV) inverted terminal repeats (ITRs).

In accordance with the present invention there may be employed conventional molecular biology, microbiology, protein expression and purification and recombinant DNA techniques within the skill of the art. Such techniques are explained fully in the literature. See, e.g., Sambrook et al. (2001)Molecular Cloning: A Laboratory Manual. 3^{rd} ed. Cold Spring Harbor Laboratory Press: Cold Spring Harbor, N.Y.; Ausubel et al. eds. (2005) Current Protocols in Molecular Biology. John Wiley and Sons, Inc.: Hoboken, N.J.; Bonifacino et al. eds. (2005) Current Protocols in Cell Biology. John Wiley and Sons, Inc.: Hoboken, N.J.; Coligan et al. eds. (2005) Current Protocols in Immunology, John Wiley and Sons, Inc.: Hoboken, N.J.; Coico et al. eds. (2005) Current Protocols in Microbiology, John Wiley and Sons, Inc.: Hoboken, N.J.; Coligan et al. eds. (2005) Current Protocols in Protein Science, John Wiley and Sons, Inc.: Hoboken, N.J.; and Enna et al. eds. (2005) Current Protocols in Pharmacology, John Wiley and Sons, Inc.: Hoboken, N.J.; Nucleic Acid Hybridization, Hames & Higgins eds. (1985); Transcription And Translation, Hames & Higgins, eds. (1984); Animal Cell Culture Freshney, ed. (1986); Immobilized Cells And Enzymes, IRL Press (1986); Perbal, A Practical Guide To Molecular Cloning (1984); and Harlow and Lane. Antibodies: A Laboratory Manual (Cold Spring Harbor Laboratory Press: 1988).

### Methods of Producing IGF-2

The disclosure provides methods of producing IGF-2, as described herein. In some embodiments, the methods comprise: (a) contacting a cell with a polynucleotide or vector comprising an IGF-2 sequence; (b) culturing said cell under conditions whereby the IGF-2 is expressed by the cell; and (c) purifying IGF-2.

In some embodiments, a host cell comprising one or more IGF-2 polynucleotides of the invention is provided. In certain embodiments, a host cell comprising one or more vectors of the invention is provided. The polynucleotides and vectors may incorporate any of the features, singly or in combination, described herein in relation to polynucleotides and vectors, respectively. In one such embodiment a host cell comprises (e.g. has been transformed or transfected with) a vector comprising a polynucleotide that encodes an amino acid sequence comprising the IGF-2 polypeptide of the invention.

As used herein, the term “host cell” refers to any kind of cellular system which can be engineered to generate the IGF-2 polypeptides, fragments, variants or derivatives thereof of the disclosure. Host cells suitable for replicating and for supporting expression of IGF-2 polypeptides are well known in the art. Such cells may be transfected or transduced as appropriate with the particular expression vector and large quantities of vector containing cells can be grown for seeding large scale fermenters to obtain sufficient quantities of the IGF-2 for clinical applications. Suitable host cells include prokaryotic microorganisms, such as E. coli, or various eukaryotic cells, such as Chinese hamster ovary cells (CHO), insect cells, or the like. For example, polypeptides may be produced in bacteria in particular when glycosylation is not needed. After expression, the polypeptide may be isolated from the bacterial cell paste in a soluble fraction and can be further purified. In addition to prokaryotes, eukaryotic microbes such as filamentous fungi or yeast are suitable cloning or expression hosts for polypeptide-encoding vectors, including fungi and yeast strains whose glycosylation pathways have been “humanized,” resulting in the production of a polypeptide with a partially or fully human glycosylation pattern. Suitable host cells for the expression of (glycosylated) polypeptides are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells. Vertebrate cells may also be used as hosts. For example, mammalian cell lines that are adapted to grow in suspension may be useful. Other examples of useful mammalian host cell lines are monkey kidney CV line transformed by SV40 (COS-7); human embryonic kidney line (293 or 293T cells as described, e.g., in Graham et al., J Gen Virol 36, 59 (1977)), baby hamster kidney cells (BHK), mouse sertoli cells (TM4 cells as described, e.g., in Mather, Biol Reprod 23, 243-251 (1980)), monkey kidney cells (CVI), African green monkey kidney cells (VERO-76), human cervical carcinoma cells (HELA), canine kidney cells (MDCK), buffalo rat liver cells (BRL 3A), human lung cells (W138), human liver cells (Hep G2), mouse mammary tumor cells (MMT 060562), MRC 5 cells, and FS4 cells. Other useful mammalian host cell lines include Chinese hamster ovary (CHO) cells, including dhfr CHO cells; and myeloma cell lines such as YO, NS0, P3X63 and Sp2/0. Host cells include cultured cells, e.g., mammalian cultured cells, yeast cells, insect cells, bacterial cells and plant cells, to name only a few, but also cells comprised within a transgenic animal, transgenic plant or cultured plant or animal tissue. In one embodiment, the host cell is a eukaryotic cell, preferably a mammalian cell, such as a Chinese Hamster Ovary (CHO) cell, a human embryonic kidney (HEK) cell or lymphoid cell (e.g., YO, NS0, Sp20 cell).

All methods of purifying IGF-2 protein following culture of IGF-2 producing cells are envisaged as within the scope of the instant disclosure. Exemplary methods include, without limitation, extraction based methods, precipitation and differential solubilization, ultracentrifugation and chromatography based methods such as affinity chromatography and ion-exchange chromatography.

### Diseases and Disorders

The disclosure provides methods of treating seizures comprising administering a therapeutically effective amount of IGF-2 or a pharmaceutical composition comprising IGF-2. Seizures treatable by the methods of the disclosure can be due to a variety of sources, discussed in further detail below.

Subjects can develop seizures due to neurodevelopmental or neurobiological diseases or disorders, additional seizure-inducing diseases or disorders, congenital abnormalities, injuries, infections and environmental conditions.

Exemplary neurodevelopmental or neurobiological diseases and disorders associated with seizures include, but are not limited to, autism spectrum disorder (ASD), Angelman Syndrome (AS), cerebral palsy, Ohtahara Syndrome, benign familial neonatal seizures, West Syndrome, Dravet Syndrome, Rett Syndrome, Tuberous sclerosis, Sturge-Weber Syndrome, Landau-Kleffner Syndrome, Lennox-Gastaut Syndrome, Rasmussen Syndrome, Gelastic Epilepsy, Benign Rolandic Epilepsy, Panayiotopoulos syndrome, Gastaut-type Syndrome, childhood absence epilepsy, and juvenile myoclonic epilepsy.

Additional diseases and disorders associated with seizures include, without limitation, autoimmune disorders such as a cerebral vasculitis and anti-NMDA receptor encephalitis; cerebral edemas such as eclampsia and hypertensive encephalopathy; cerebral ischemia or hypoxia such as cardiac arrhythmias, carbon monoxide toxicity, nonfatal drowning, near suffocation, stroke and vasculitis; expanding intracranial lesions, such as lesions due to hemorrhage, hydrocephalus, and tumors; metabolic disorders such as hypocalcemia, hypoglycemia, hyponatremia, hepatic encephalopathy, uremic encephalopathy, hyperglycemia, hypomagnesemia, hypernatremia and vitamin B6 deficiency; and neurocutaneous disorders such as neurofibromatosis and tuberous sclerosis. In some embodiments, the seizures are caused by a brain tumor.

Seizures can be caused by genetic disorders, for example lipid-storage diseases such as Tay-Sachs disease, neuronal migration disorders such as heterotopias, and phenylketonuria. In some embodiments, the disease or disorder comprises Doose syndrome, CDKL5 disorder, pediatric epilepsy related to PCDH19, Dravet syndrome, Angelman syndrome, SCN1A-related epilepsy, Ohtahara syndrome, Glut1 Deficiency Syndrome and TBCK-related ID Syndrome.

Seizures can also be caused by injuries, commonly head dramas. Exemplary head dramas include birth injuries, as well as blunt and penetrating injuries. For example, posttraumatic seizures occur in 25 to 75% of patients who have brain contusion, skull fracture, intracranial hemorrhage, prolonged coma, or focal neurologic deficits. Further injuries that can cause seizures include pressure related injuries, such as decompression illness and hyperbaric oxygen treatments.

Seizures can be caused by infections of the central nervous system. Exemplary infections that can cause seizures include AIDS, brain abscess, Falciparum malaria, meningitis, neurocysticercosis, neurosyphilis, rabies, tetanus, toxoplasmosis, and viral encephalitis.

Hyperpyrexia due to drug toxicity (cocaine, or amphetamines, e.g.), fever or heatstroke can also lead to seizures. Withdrawal symptoms, such as withdrawal from alcohol, anesthetics, barbituates, or benzodiazepenes can include seizures.

Seizures can be caused by environmental factors. For example, heavy metal poisoning and herbicides, as well as a variety of medications and recreational drugs can induce seizures. Antidepressants, diphenhydramine, stimulants (including cocaine and methamphetamine), tramadol and isoniazid are all associated with seizures.

In some embodiments, the subject is diagnosed with epilepsy. Epilepsy describes a central nervous system (neurological) disorder in which brain activity becomes abnormal, causing seizures or periods of unusual behavior, sensations, and sometimes loss of awareness. In some embodiments, the epilepsy comprises epilepsy, epilepsy with generalized tonic-clonic seizures, epilepsy with myoclonic absences, frontal lobe epilepsy, temporal lobe epilepsy, occipital lobe epilepsy, parietal lobe epilepsy, juvenile myoclonic epilepsy (JME), intractable childhood epilepsy (ECI), childhood absence epilepsy, Benign Rolandic epilepsy, status epilepticus, refractory status epilepticus, status super refractory epilepticus (SRSE), or pediatric epilepsy related to PCDH19.

In some embodiments, the neurodevelopmental or neurological disorder comprises autism spectrum disorder (ASD) or Angelman Syndrome (AS).

Angelman Syndrome is a neurogenetic disorder characterized by intellectual and developmental delay. AS is caused mostly by deletions of the maternal allele in the region 15q11-q13 which includes the E3 ubiquitin ligase Ube3A. This region is paternally imprinted and approximately 2% of cases result from paternal uniparental disomy of 15q11.2-q13; and 2 to 3% result from imprinting defects. A subset of the remaining 25% are caused by mutations in the gene encoding the ubiquitin protein ligase E3A gene (UBE3A). Symptoms of Angelman Syndrome can include: developmental delays such as a lack of crawling or babbling at 6 to 12 months, attenuated mental development, no or minimal speech, ataxia (inability to move, walk, or balance properly), stiff or jerky movements (e.g., hand-flapping), hyperactivity, trembling in the arms and legs, frequent smiling and laughter, bouts of inappropriate laughter, widely spaced teeth, a happy, excitable personality, epilepsy, an electroencephalographic abnormality with slowing and notched wave and spikes, seizures which usually begin at 2 to 3 years of age and may be accompanied by myoclonus and atypical absence, partial seizures with eye deviation and vomiting, a small head which is noticeably flat in the back (microbrachyoephaly), crossed eyes (strabismus), thrusting of the tongue and suck/swallowing disorders, protruding tongue, excessive chewing/mouthing behaviors, hyperactive lower extremity deep tendon reflexes, wide-based gait with pronated or valgus-positioned ankles, increased sensitivity to heat, walking with the arms up in the air, fascination with water or crinkly items such as some papers or plastics, obesity in older children, constipation, a jutting lower jaw, light pigmentation of the hair, skin, and eyes (hypopigmentation), frequent drooling, prognathia, feeding problems and/or truncal hypotonia during infancy, and/or scoliosis. Symptoms are usually not evident at birth and are often first evident as developmental delays such as a failure to crawl or babble between the ages of 6 to 12 months as well as slowing head growth before the age of 12 months. Individuals with Angelman Syndrome may also suffer from sleep disturbances including difficulty initiating and maintaining sleep, prolonged sleep latency, prolonged wakefulness after sleep onset, high number of night awakenings and reduced total sleep time, enuresis, bruxism, sleep terrors, somnambulism, nocturnal hyperkinesia, and snoring.

Severity of symptoms for AS can be measured clinically (Williams et al., American Journal of Medical Genetics 2005 140A; 413-8, incorporated herein by reference) and quantification of the severity of different symptoms can also be carried out (Lossie et al., Journal of Medical Genetics 2001, 38; 834-845, incorporated herein by reference; Ohtsuka et al., Brain and Development 2005, 27; 95-100, incorporated herein by reference). This may include the extent of language ability, degree of independent mobility, frequency and severity of seizures, ability to comprehend language, acquisition of motor skills, growth parameters. A screening procedure for suspected Angelman syndrome patients that quantifies the severity of 22 distinct criteria (Lossie et al., Journal of Medical Genetics 2001, 38) can be used. Other measurements of AS severity include psychometric methods to distinguish the degree of developmental delay with respect to psychomotor developmental achievement, visual skills, social interactions based on non-verbal events, expressive language abilities, receptive language abilities, and speech impairment. The degree of gait and movement disturbances as well as attention ability and the extent of EEG abnormalities can be measured (Williams et al., American Journal of Medical Genetics 2005 140A; 413-8). At appropriate age intellectual ability tests can also be used, such as the Kaufman Brief Intelligence Test-2 (KBIT-2; Kaufman & Kaufman, Circle Pines, Minn.: American Guidance Services; 2004, incorporated herein by reference). One or more of the above characteristics can be used to evaluate the effectiveness of treatment with the present compositions.

In an embodiment, assessment protocols to evaluate the effect of treatment include neurological and neurovisual examination and the evaluation of motor (e.g. Gross Motor Function Measure Scale), cognitive (e.g. Griffiths Mental Development Scale and Uzgiris-Hunt Scale and spatial working memory tests); adaptive (e.g. Vineland Adaptive Behavioral Scale); communication (e.g. MacArthur-Bates Communicative Development Inventory and video-recordings children's verbal expression), behavioral aspects (e.g. IPDDAG Scale) and neurovisual aspects as described in Micheletti et al., (Ital J Pediatr. 2016; 42(1): 91), incorporated herein by reference.

Autism spectrum disorders are characterized by complex developmental disability that interferes with the normal development of the brain, particularly impacting social interaction and communication skills. It typically appears during the first three years of life. Autistic individuals have difficulties in verbal and non-verbal communication, social interactions, and leisure or play activities. Impairment in social interaction range from difficulty initiating and maintaining interaction, impaired ability to recognize and experience emotions, and difficulty processing and appreciating the feelings of others. Communication deficits vary amongst autistic individuals, with some autistic individuals having severely limited form of communication to individuals having significant language skills. Repetitive and stereotypic behaviors include complex rituals, difficulty in adapting to change, and unusual movements such as hand flapping. Some characteristic behavior which may be useful for diagnosing autism includes lack of or delay in spoken language, repetitive use of language or motor mannerisms (hand flapping or twirling objects), little or no eye contact, persistent fixation on certain objects, and lack of interest in socializing.

Any sort of seizure is envisaged as within the scope of the instant disclosure. Seizures include focal seizures, and generalized seizures. In some embodiments, the focal seizures comprise focal seizures without loss of consciousness or focal seizures with impaired awareness. In some embodiments, the generalized seizures comprise absence seizures, tonic seizures, atonic seizures, clonic seizures, myoclonic seizures, or tonic-clonic seizures.

Focal seizures, sometimes called partial seizures, result from abnormal activity in just one area of the brain. There are two categories of focal seizures: focal seizures without loss of consciousness, and focal seizures with impaired awareness. In focal seizures without loss of consciousness, the seizures do not cause a loss of consciousness, but instead may alter emotions or change sensory perception, such as the way things look, smell, feel or taste. Focal seizures without loss of consciousness may also results in in involuntary jerking of a body part, such as an arm or leg, and spontaneous sensory symptoms such as tingling, dizziness and flashing lights. In focal seizures with impaired awareness, sometimes called complex partial seizures, the seizures involve a change or loss of consciousness or awareness. During the seizure, the subject may stare into space and not respond normally to the environment, or perform repetitive movements, such as hand rubbing, chewing, swallowing or walking in circles.

Generalized seizures are seizures that involve all areas of the brain, and fall into six categories: absence seizures, tonic seizures, atonic seizures, clonic seizures, myoclonic seizures and tonic-clonic seizures. Absence seizures, sometimes called petit mal seizures, are characterized by staring into space, brief loss of awareness, and subtle body movements such as eye blinking or lip smacking. These seizures are common in children. Tonic seizures cause stiffening of the muscles, usually muscles of the back, arms and legs, and may cause falling. Atonic seizures, sometimes also called drop seizures, cause a loss of muscle control, which may also cause falling. Clonic seizures are associated with repeated or rhythmic, jerking muscle movements, and usually affect the neck, face and arms. Myoclonic seizures are characterized by sudden brief jerks or twitches of arm and leg muscles. Tonic-clonic seizures, also called grand mal seizures, can cause an abrupt loss of consciousness, body stiffening and shaking, and sometimes loss of bladder control and tongue biting.

In some embodiments, the seizures are audiogenic seizures. Audiogenic seizures are seizures that are triggered acoustic stimulation. Audiogenic seizures are a type of reflex epilepsy, i.e. epilepsy in which environmental stimulus triggers seizures. Common seizure types associated with reflex epilepsy include tonic-clonic, absence, myoclonic, and focal seizures.

The subject with seizures can be an infant, a child or an adult.

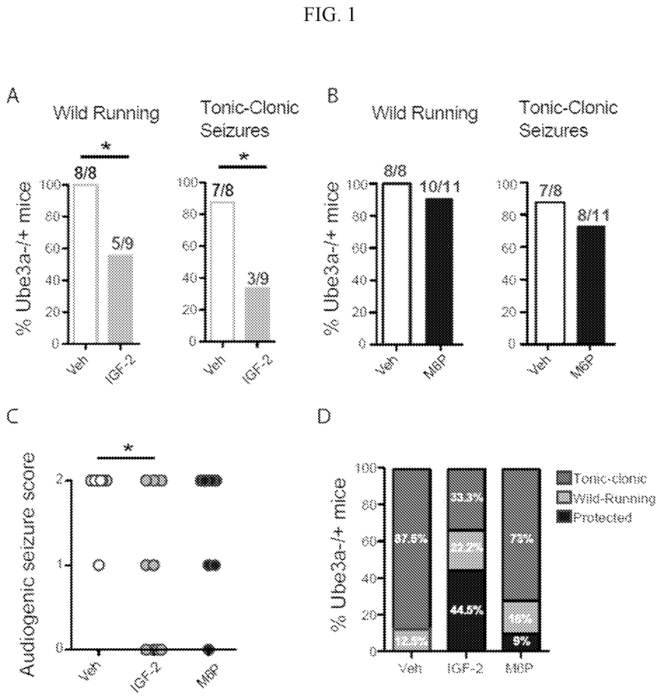

As used herein, “treating a seizure” refers to a partial or full reduction of the severity, duration, or frequency of a seizure, or one or more symptoms associated with the seizure. Treatment may improve one or more symptoms of a seizure. Effective treatment of a seizure disorder can be established by presenting a reduction in the frequency, severity or duration of seizures (e.g., more than 10%, 20%, 30% 40%, 50% or more) after a period of time, compared to baseline. For example, after a baseline period of one month, subjects administered IGF-2 may be compared to an equivalent group of subjects administered a vehicle only control in a double-blinded study, and the effect of IGF-2 administration may be measured using the methods described in the Examples (see, for example, FIGS. 1A-1D and associated methods). The skilled artisan will appreciate that treatment encompasses both complete prevention and reversal of seizures, as well as reduction in frequency, severity or duration of seizures, or the reduction of one or more symptoms associated with the seizures. Reduction of frequency, severity or duration that is less than complete prevention can nonetheless be biologically and clinically relevant, and of therapeutic benefit. For example, treatment with the IGF-2 compositions of the disorder may prevent loss of consciousness during a seizure or reduce the duration of loss of consciousness, prevent or reduce muscle spasms associated with a seizure, prevent or reduce repetitive movements associated with a seizure, or a combination thereof.

As used herein, “preventing” or “reversing” seizures refers to a subject who would otherwise be expected to suffer from seizures, who nonetheless does not have seizures following administration of the IGF-2 compositions of the disclosure. For example, an individual diagnosed with a genetic disease that causes seizures, or who has a history of seizures who then does not have seizures following administration of the IGF-2 compositions of the disorder can be said to have their seizures prevented or reversed.

### Dosage and Administration

Administration of IGF-2, IGF-2 modifications (e.g., IGF-2 analogs including IGF-2 peptides or derivatives) are within the scope of the instant disclosure. IGF-2 peptides, as well as other analogs or modifications thereof are disclosed in U.S. Application Publication No. 20120266263, incorporated herein by reference. The frequency and length of treatment can be determined by monitoring one or more symptoms of AS or ASD. The treatment can be continued as long as needed, including days, months, or years. The treatment can be continued even after the symptoms have subsided or no longer measurable.

Generally, a therapeutic dose of IGF-2 for the present disclosure is in the range of about 1 to 10,000 microgram/kg of the subject's body weight. IGF-2 may be used at from about 1 to 500 μg/kg of the subject's body weight and all values and ranges therebetween. For example, IGF-2 may be used at 1 to 500 μg/kg, 1 to 100 μg/kg, 1 to 50 μg/kg, 10 to 500 μg/kg, 10 to 100 μg/kg, 10 to 50 μg/kg of the subject's body weight. In one embodiment, IGF-2 can be used at 10 to 45 μg/kg of the subject's body weight administered subcutaneously. In specific embodiments, IGF-2 can be used at 1, 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, and 500 μg/kg of the subject's body weight.

In an embodiment, this disclosure provides a method for treatment of neurodevelopmental disorders characterized by seizures. In some instances, seizures can be seen by about toddler age. Examples of such neurodevelopmental disorders include Angelman syndrome and autism spectrum disorders.

In an embodiment, the present disclosure provides a method for preventing, treating and/or reversing epileptic seizures associated with Angelman Syndrome or autism spectrum disorders. The epileptic seizures may be audiogenic seizures (induced by sound). The method comprises administering to an individual in need of treatment a therapeutically effective amount of IGF-2, for example, a dose of IGF-2 in the range of about 1 to 10,000 microgram/kg of the subject's body weight. IGF-2 may be used at from about 1 to 500 μg/kg of the subject's body weight and all values and ranges therebetween. For example, IGF-2 may be used at 1 to 500 μg/kg, 1 to 100 μg/kg, 1 to 50 μg/kg, 10 to 500 μg/kg, 10 to 100 μg/kg, 10 to 50 μg/kg of the subject's body weight. In an embodiment, IGF-2 can be used at 10 to 45 μg/kg of the subject's administered subcutaneously. In specific embodiments, IGF-2 can be used at 10, 15, 20, 25, 30, 35, 40, 45, 50, 55, 60, 65, 70, 75, 80, 85, 90, 95, 100, 200, 300, 400 and 500 μg/kg of the subject's body weight. In specific embodiments, IGF-2 can be used at 1, 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, and 500 μg/kg of the subject's body weight.

Administration of present compositions can be carried out using any suitable route of administration known in the art. For example, the compositions may be administered via intravenous, intramuscular, intraperitoneal, intracerebrospinal, subcutaneous, intra-articular, intrasynovial, oral, topical, or inhalation routes. The compositions may be administered parenterally or enterically. In one embodiment, the compositions of the present disclosure can be administered orally, such as, for example, in the form of a tablet, capsule, pill, powder, paste, granules, elixir, solution, suspension, dispersion, gel, syrup or any other ingestible form. IGF-2 and/or modifications (e.g., IGF-2 analogs) thereof may be delivered via liposomes, microparticles, microcapsules. The compositions may be introduced as a single administration or as multiple administrations or may be introduced in a continuous manner over a period of time. For example, the administration(s) can be a pre-specified number of administrations or daily, weekly or monthly administrations, which may be continuous or intermittent, as may be clinically needed and/or therapeutically indicated.

In an embodiment, the IGF-2 and/or its derivative or analog is the only active component.

Various delivery systems are known and are used to administer a therapeutic of the invention, e.g., encapsulation in liposomes, microparticles, microcapsules, expression by recombinant cells, receptor-mediated endocytosis (see, e.g., Wu & Wu, J. Biol. Chem. 265:4429-4432, 1987), construction of a therapeutic nucleic acid as part of a viral or other vector, etc.

In some embodiments, the methods of treating seizures comprise administering a nucleic acid encoding an IGF-2. In some embodiments, the nucleic acid can be administered in vivo to promote expression of IGF-2, by constructing it as part of an appropriate nucleic acid expression vector and administering it so that it becomes intracellular, e.g., by use of a viral vector (see U.S. Pat. No. 4,980,286), or by direct injection, or by use of microparticle bombardment (e.g., a gene gun; Biolistic, Dupont) or coating with lipids or cell-surface receptors or transfecting agents, or by administering it in linkage to a homeobox-like peptide that is known to enter the nucleus (see, e.g., Joliot et al. (1991). Proc. Natl. Acad. Sci., U.S.A. 88, pp. 1864-1868). Exemplary, but non-limiting vectors include lentiviral vectors and adeno-associated viral vectors.

Alternatively, a nucleic acid therapeutic can be introduced intracellularly and incorporated within host cell DNA for expression by homologous recombination. For example, a donor template nucleic acid comprising IGF-2 operably linked to a suitable promoter can be introduced into a host cell using homology directed repair (HDR) mechanisms. Double strand breaks that are repaired by host cell endogenous DNA repair machinery can be introduced by any suitable means known in the art, including CRISPR/Cas9, zinc-finger endonucleases and Transcription activator-like effector nuclease (TALENs).

Other methods for improving the delivery and administration of the pharmacological agent of the present invention include means for improving the ability of the pharmacological agent to cross membranes, and in particular, to cross the blood-brain barrier. One skilled in the art can readily assay the ability of IGF-2 to cross the blood-brain barrier in vivo, for example using a model of the blood-brain barrier based on a brain microvessel endothelial cell culture system (see, e.g., Bowman et al., (1983). Ann. Neurol. 14, pp. 396-402; Takahura et al. (1992). Adv. Pharmacol. 22, 137-165). In one embodiment, the IGF-2 can be modified to improve its ability to cross the blood-brain barrier, and in an alternative embodiment, the IGF-2 can be co-administered with an additional agent, such as for example, an anti-fungal compound, that improves the ability of the pharmacological agent to cross the blood-brain barrier (see Pardridge (2002). W. M. Neuron 36, pp. 555-558).

### Pharmaceutical Compositions

The agents of the present disclosure, i.e., IGF-2 and functionally modified derivatives thereof, can be administered to subjects in pharmaceutical compositions by combining the agent with one or more suitable pharmaceutically acceptable carriers, excipients and/or stabilizers.

The pharmaceutical compositions of the present invention include peptides, proteins and nucleic acid molecules, and may be formulated for administration in any convenient way for use in human or veterinary medicine. The invention therefore includes within its scope pharmaceutical compositions comprising a compound of the invention adapted for use in human or veterinary medicine.

Examples of pharmaceutically acceptable carriers, excipients and stabilizer can be found in Remington: The Science and Practice of Pharmacy (2005) 21st Edition, Philadelphia, Pa. Lippincott Williams & Wilkins.

Excipients may be selected from the group consisting of antioxidants (e.g., hindered phenols (e.g., tetrakis [methylene (3,5-di-t-butyl-4-hydroxyhydrocinnamate)]methane), less-hindered phenols, and semi-hindered phenols; phosphates, phosphites, and phosphonites (e.g., tris (2,4-di-t-butylphenyl) phosphate); thio compounds (e.g., distearyl thiodipropionate, dilaurylthiodipropionate); various siloxanes; and various amines (e.g., polymerized 2,2,4-trimethyl-1,2-dihydroquinoline), non-aggregating agents/lubricants (e.g., boric acid, PEG4000, PEG6000, sodium oleate, sodium benzoate, sodium acetate, sodium acetate, sodium stearate, sodium stearyl fumarate, sodium lauryl sulfate, magnesium lauryl sulfate, magnesium stearate, stearate, stearic acid, talc, hydrogenated oil, and glyceryl behenate), binding agents (e.g., acacia, gelatin, starch paste, polyvinylpyrrolidone, polyethylene glycol, glucose, carboxymethyl cellulose, and povidone), fillers/diluents (e.g., starches or partially gelatinized starches, sorbitol, mannitol, malitol, microcrystalline cellulose); disintegrants (e.g., sodium croscarmellose and sodium starch glycolate); plasticizers (e.g., glycerol, vitamin E TPGS, triacetin); anti-tacking agents (e.g., tricalcium phosphate, silicon dioxide, bentonite); wetting agents (sodium lauryl sulfate, sodium stearyl fumarate, polyoxyethylene 20 sorbitan monooleate (e.g., Tween™); sweeteners (sucralose, sorbitol, and xylitol); colorants (FD&C Blue #1 Aluminum Lake, FD&C Blue #2, other FD&C Blue colors, titanium dioxide, iron oxide); flavorants (menthol, peppermint oil, almond oil); glidants (colloidal silica, precipitated silica, and talc); pH adjusters (arginine, tartaric acid, sodium hydrogen carbonate, adipic acid); and surfactants (ammonium lauryl sulfate, sodium lauryl sulfate (sodium dodecyl sulfate, SLS, or SDS), sodium laureth sulfate, sodium myreth sulfate, dioctyl sodium sulfosuccinate, fatty acid esters of glycerol, poloxamers).

In one embodiment, suitable carriers include excipients, or stabilizers which are nontoxic to recipients at the dosages and concentrations employed, and include buffers such as acetate, Tris, phosphate, citrate, and other organic acids; antioxidants including ascorbic acid and methionine; preservatives such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol; amino acids such as glycine, glutamine, asparagine, histidine, arginine, or lysine; monosaccharides, disaccharides, and other carbohydrates including glucose, mannose, or dextrins; chelating agents such as EDTA; tonicifiers such as trehalose and sodium chloride; sugars such as sucrose, mannitol, trehalose or sorbitol; surfactant such as polysorbate; salt-forming counter-ions such as sodium; and/or non-ionic surfactants such as Tween or polyethylene glycol (PEG).

In one embodiment, the antioxidant is selected from the group consisting of distearyl thiodipropionate, dilauryl thiodipropionate, octadecyl-3,5-di-t-butyl-4-hydroxyhydrocinnamate, benzenepropanoic acid, 3,5-bis (1,1-dimethylethyl)-4-hydroxy-thiodi-2,1-ehtanediyl ester, stearyl 3-(3,5-di-t-butyl-4-hydroxyphenyl) propionate, octadecyl-3-(3,5-di-tert-butyl-4-hydroxyphenyl)-propionate, 2,4-bis(dodecylthiomethyl)-6-methylphenol, 4,4′-thiobis(6-tert-butyl-m-cresol), 4,6-bis (octylthiomethyl)-o-cresol, 1,3,5-tris(4-tert-butyl-3-hydroxy-2,6-dimethyl benzyl)-1,3,5-triazine-2,4,6-(1H,3H,5H)-trione, pentaerythritol tetrakis (3-(3,5-di-t-butyl-4-hydroxyphenyl)propionate), 2′,3-bis[[3-[3,5-di-tert-butyl-4-hydroxyphenyl]propionyl]]propionohydrazide), buffering agents (e.g., borates, borate-polyol complexes, succinate, phosphate buffering agents, citrate buffering agents, acetate buffering agents, carbonate buffering agents, organic buffering agents, amino acid buffering agents), bulking agents/fillers (e.g., lactose monohydrate, microcrystalline cellulose, cellulose acetate, calcium carbonate, potato starch, sucrose, sorbitol, and dextrose).

Compositions of the invention may also include dyes, coatings, and/or preservatives.

Examples of suitable pharmaceutically acceptable dyes useful for the compositions of the present invention include, but are not limited to, synthetic and natural dyes such as titanium dioxide, beta-carotene and extracts of grapefruit peel.

Examples of pharmaceutically acceptable coatings useful for the oral compositions of the present invention, typically used to facilitate swallowing, modify the release properties, improve the appearance, and/or mask the taste of the compositions include, but are not limited to, hydroxypropylmethylcellulose, hydroxypropylcellulose and acrylate-methacrylate copolymers.

Suitable examples of pharmaceutically acceptable preservatives include, but are not limited to, various antibacterial and antifungal agents such as solvents, for example ethanol, propylene glycol, benzyl alcohol, chlorobutanol, quaternary ammonium salts, and parabens (such as methyl paraben, ethyl paraben, propyl paraben, etc.).

The pharmaceutical compositions of the present disclosure contain IGF-2, or a functionally modified derivative thereof, in an amount from 0.01 to 99% weight per volume or weight per weight.

The compositions may be administered orally, intranasally, topically or anally. Additionally, the compositions presented herein can be administered via a parenteral route. For example, the compositions can be administered by a route selected from intramuscular, intraperitoneal, intravenous and intracerebral, or any combination thereof.

In a further embodiment, the compositions of the present invention are administered by a route selected from the group consisting of intranasal, oral, topical, anal, ocular, optic, intramuscular, intraperitoneal, intravenous and intracerebral, or any combination thereof.

In some embodiments, the compositions comprising IGF-2 of the disclosure are administered parenterally. In some embodiments, the parenteral administration comprises intramuscular, intraperitoneal, intravenous, subcutaneous, intrathecal, intraspinal or intracerebral administration.

The compositions used in the invention may be milled using known milling procedures such as wet milling to obtain a particle size appropriate for tablet formation and for other formulation types. Finely divided (nanoparticulate) preparations of the compounds may be prepared by processes known in the art, for example see International Patent Application No. WO 02/00196 (SmithKline Beecham).

The compositions of the present invention can be administered orally (e.g., as a tablet, sachet, capsule, pastille, pill, bolus, powder, paste, granules, bullets or premix preparation, ovule, elixir, solution, suspension, dispersion, gel, syrup or as an ingestible solution).

Additionally, the compositions presented herein can be formulated for parenteral administration (e.g., intramuscular, intraperitoneal, intravenous, intracerebral). Compounds may be present as a dry powder for constitution with water, PBS, or other suitable vehicle before use, optionally with flavoring and coloring agents. Solid and liquid compositions may be prepared according to methods well-known in the art. Such compositions may also contain one or more pharmaceutically acceptable carriers and excipients which may be in solid or liquid form.

Dispersions can be prepared in a liquid carrier or intermediate, such as glycerin, liquid polyethylene glycols, triacetin oils, and mixtures thereof. The liquid carrier or intermediate can be a solvent or liquid dispersive medium that contains, for example, water, ethanol, a polyol (e.g., glycerol, propylene glycol or the like), vegetable oils, non-toxic glycerine esters and suitable mixtures thereof. Suitable flowability may be maintained, by generation of liposomes, administration of a suitable particle size in the case of dispersions, or by the addition of surfactants.

The tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycolate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia.

### Kits and Articles of Manufacture

The disclosure provides kits comprising IGF-2, functionally modified derivatives thereof, or pharmaceutical compositions comprising the same, along with instructions for use. The following examples are provided as illustrative examples and are not intended to be restrictive in any way.

### EXAMPLES

### Example 1: Effect of IGF-2 on Seizures

This example demonstrates that insulin-like growth factor 2 protects from seizures.

Results and Discussion

IGF-2, but not M6P, Attenuates Audiogenic Seizures of Ube3a^{m-/p+} Mice

Eighty to ninety-five % of individuals with AS have seizures (Fiumara et al., 2010, Thibert et al., 2013). Similarly, Ube3a^{m-/p+} mice are highly susceptible to audiogenic seizures, a phenotype that is specifically observed in mice in the 129S2 background (Born et al., 2017). We therefore generated a colony of Ube3a^{m-/p+} mice in the 129S2 background (Jackson Labs stock #004477) to test whether IGF-2 or M6P affected audiogenic-induced seizures. A very robust seizure phenotype was detected in 100% of these animals, as assessed using protocols of audiogenic seizures previously established (Mandel-Brehm et al., 2015; Sonzogni et al., 2018). A s.c. injection of IGF-2 twenty minutes prior the audiogenic stimulus significantly decreased the frequency and severity of seizures relative to vehicle alone: wild running was reduced by approximately 44% and tonic-clonic seizures by approximately 67%. In contrast, injection of M6P had no effect (FIGS. 1A-D).

It remains to be determined whether or not M6P at other doses might be effective on seizures. Based on the present data, we cannot exclude the possibility that the two ligands produce differential effects, which may result from differences in their binding and biological properties. For example, the effects of IGF-2 on seizures may occur via another type of receptor.

Acute IGF-2 or M6P treatment did not produce adverse effects in Ube3a^{m-/p+} mice. To determine whether the IGF-2 or M6P produces adverse effects, we assessed the safety of the single s.c. IGF-2 and M6P injection by conducting a standard observational battery of tests that included physical, behavioral, and sensorimotor evaluations (Paylor et al., 1998). As shown in Table 1, no differences were found between WT and Ube3a^{m-/p+} mice treated with vehicle, IGF-2, or M6P mice at 30 min, 24 h, or 1 week after injection. We concluded that acute systemic treatment with IGF-2 and M6P in Ube3a^{m-/p+} mice does not have any major aversive effect.

To our knowledge, there is no previous report of potential therapeutic agents in AS model mice that encompass multiple cognitive and motor behaviors, except for approaches that target the unsilencing of the paternal allele (Jamal et al., 2017, Kaphzan et al., 2012, Meng et al., 2013, Sun et al., 2016), These treatments however have not shown to be effective in controlling seizures (Yang, 2019).

Methods

Male and female mice were used for experiments at equal genotypic ratio. In all the experiments, both males and females were included and analyzed as a single group, because statistical analyses of separate sex groups, although with small n, therefore preliminary (n=2-8), yielded no significant difference (unpaired two-tailed Student's t test, P>0.05). All experiments and analyses were performed blind to genotype and treatment, and all statistical analyses were performed using GraphPad Prism software (GraphPad Software Inc.) with significance set at P<0.05. Multi-group comparisons were analyzed using one-way or two-way ANOVA with Tukey's post hoc test or Kruskal-Wallis test with Dunn's multiple comparisons. Chi-square test was used to compare the percent of seizures between the two treatments used in AS mice. All procedures complied with the US National Institute of Health Guide for the Care and Use of Laboratory Animals and were approved by the New York University Animals Care Committees.

Mice

The studies were performed on wild-type (WT) male and female mice and ubiquitin protein ligase E3A (Ube3A) (m−/p+) male and female young adult mice (8 to 12 weeks) in the B6.12952 background. To establish the colony, male mice carrying a paternally imprinted Ube3A knockout mutation (Jackson labs www.jax.org; stock #004477) were paired with C57 female mice. Subsequent female heterozygous mice were bred with male C57 mice to obtain Ube3a^{m-/p+} mice (heterozygous males and females with maternal transmission), wild type (WT) males, and WT females. WT male and female littermates were used as controls. For audiogenic seizure experiments, we established a colony from cryopreserved Ube3a^{m-/p+} mice on a 129S2 background (Jackson labs stock #004477). Equal genotype ratio of male and female mice was used for experiments and mice were randomly assigned to experimental groups. All animal procedures were approved by the Institutional Animal Care and Use Committee of the New York University and were performed in accordance with the guidelines of the U.S. National Institutes of Health. Mice were group-housed on a 12:12 light/dark cycle with ad libitum access to food and water.

In all experiments, mice were handled for 2-3 min/d for 5 days before their first behavioral procedure. Mice were randomly assigned to receive either IGF-2, or M6P, or vehicle at the first session, after which the treatment was counterbalanced. Seven to 10 days elapsed between subsequent behavioral procedure. For all behavioral experiments, multiple repetitions each consisting of n=3-4/group conducted in different days and performed by different experimenters were carried out for data confirmation, with all treatment conditions always conducted in parallel.

Ligands

Recombinant mouse IGF-2 (R&D Systems, cat #792-MG-050) was dissolved in 0.1% BSA-PBS (sterile, pH 7.4), and injected at 30 μg/kg subcutaneously (s.c.) in 0.3 ml (Stern et al. 2014, Steinmetz et al. 2018). 850 μg/kg Mannose-6-phosphate (M6P; Sigma) was dissolved in sterile phosphate buffered saline (PBS, pH 7.4) the day of the experiment and administered s.c. This M6P dose was chosen after a dose response curve of 25, 250, 850 and 1500 μg/kg s.c. Mice were injected 20 min before induction of audiogenic seizures.

Audiogenic Seizures

An independent line of Ube3a^{m-/p+} mice in 129S2 background was established for these experiments. To induce audiogenic seizures in these Ube3a^{m-/p+} mice we used a slight modification of a standard protocol used by (Sonzoni et al., 2018). Specifically, Ube3a^{m-/p+} mice were habituated to the testing environment (polycarbonate cage) for 2 min. Audiogenic seizures were induced by vigorously scraping scissors across the metal grating of the cage lid (which creates approximately a 100-dB sound) approximately 25 cm above the test subject for 60 sec [Jiang et al. 1998; Silva-Santos et al. 2015; Sonzogni et al., 2018]. The stimulus lasted 60 seconds or terminated earlier if a tonic-clonic seizure was observed. The manifestation of seizures generally occurred in two stages. First, mice exhibited wild involuntary seizure-like movements, also known as a “wild running” bout. In most cases the wild running progresses into a more severe tonic-clonic seizure with extension of extremities. We measured seizure phenotypes by quantifying the presence of wild running and tonic-clonic convulsions on video recordings. Scoring was done by experimenters blind to genotype and treatment. The response of each mouse was scored on the basis of the most severe phenomenon seen (i.e., no seizure response=0; wild running and jumping=1; tonic-clonic seizure=2), and a mean seizure severity score was calculated.

Observational Battery

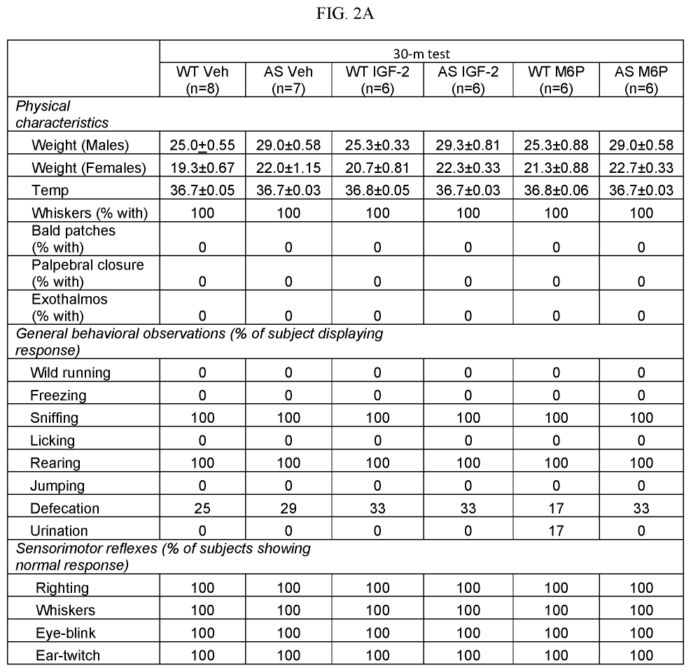

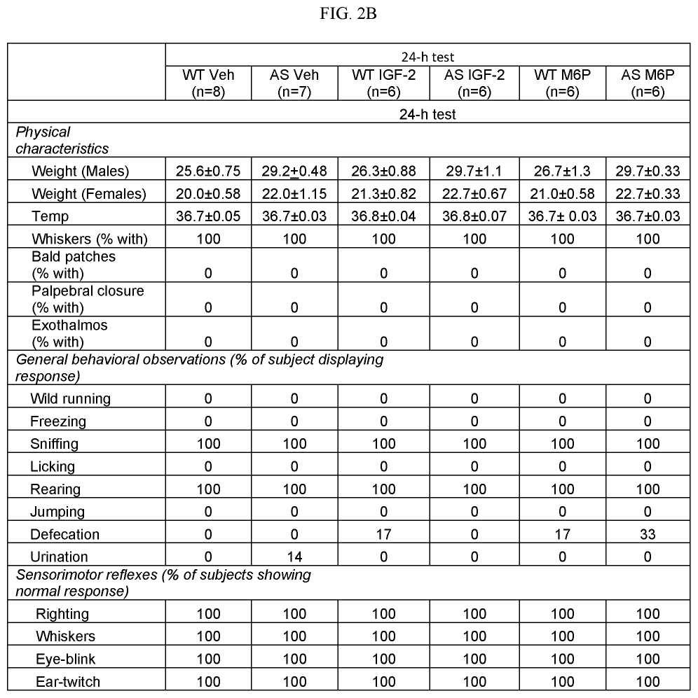

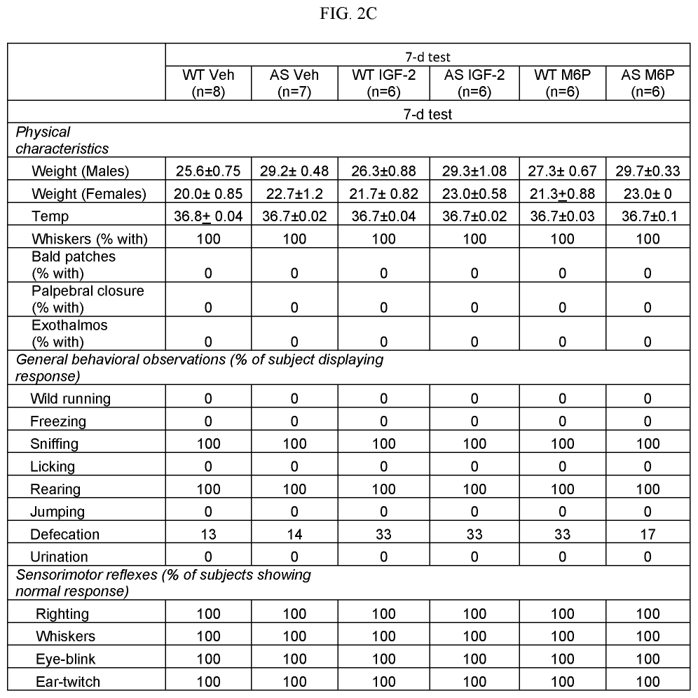

To determine whether IGF-2 or M6P causes adverse effects, we assessed the safety of s.c. injection of each drug using a standard observational battery of tests that included physical, behavioral, and sensorimotor evaluations [Brown, Jones, & Forbes, 2009]. Body temperature was taken with a digital rectal probe, and physical characteristics were recorded. Each mouse was then observed in an empty cage for 1 min, and general behavioral observations were recorded. Sensorimotor reflexes and simple motor responses were then tested in the order described in FIGS. 2A-2C. Observers blind to experimental procedures scored all experiments. As shown in FIGS. 2A-C, no differences were observed between WT and Ube3a^{m-/p+} mice treated with vehicle, IGF-2, or M6P mice at 30 min, 24 hr, or 7 days after injection. We concluded that acute systemic treatment with IGF-2 or M6P has no major aversive effect.

Observational battery was carried out as previously described (Stern et al., 2014) at designated time points after injection (30 min, 24 h and 1 week). Body temperature was taken with a digital rectal probe, and physical characteristics were recorded. Each mouse was then observed in an empty cage for 1 min, where general behavioral observations were recorded. Sensorimotor reflexes and simple motor responses were then tested in the order described in Table 1. An observer blind to experimental procedures scored all of the experiments.

Statistics

All statistical analyzes were performed using GraphPad Prism software (GraphPad Software Inc.). Two-group comparisons were analyzed using unpaired t-test; multi-group comparisons were analyzed using two-way ANOVA with Tukey's post-hoc test. We used Kruskal-Wallis to compare three or more groups on a dependent variable that is measured on an ordinal level. This statistical test has been used in previous publications with the same type of data sets [Forcelli, Kalikhman, & Gale, 2013; Gu et al., 2019]. The chi-square test was used to compare the percent of seizures between two groups. P<0.05 was considered significant. In cases where our analysis did not show significant interaction effect but had significant main effects (differences between groups or treatment), the post-hoc analysis was performed on the main effects. This is a common approach when comparing treatments at different time points [Wei, Carroll, Harden, & Wu, 2012].

### REFERENCES

Born H. A., Dao A T, Levine A T, Lee W L, Mehta N M, Mehra S, Weeber E J, Anderson A E (2017) Strain-dependence of the Angelman Syndrome phenotypes in Ube3a maternal deficiency mice. Sci Rep. 17(1), 8451.
Brown, J., Jones, E. Y., & Forbes, B. E. (2009). Keeping IGF-II under control: Lessons from the IGF—II-IGF2R crystal structure. Trends in Biochemical Sciences, 34(12), 612-619.
Forcelli, P. A., Kalikhman, D., & Gale, K. (2013). Delayed effect of craniotomy on experimental seizures in rats. PLoS One, 8, e81401.
Fiumara A, Pittala A, Cocuzza M, Sorge G (2010) Epilepsy in patients with Angelman syndrome. Ital J Pediatr 36:31 Jamal I, Kumar V, Vatsa N, Shekhar S, Singh B K, Sharma A, Jana N R (2017) Rescue of altered HDAC activity recovers behavioural abnormalities in a mouse model of Angelman syndrome. Neurobiol Dis 105:99-108.
Gu, B., Zhu, M., Glass, M. R., Rougié, M., Nikolova, V. D., Moy, S. S., Philpot, B. D. (2019). Cannabidiol attenuates seizures and EEG abnormalities in Angelman syndrome model mice. Journal of Clinical Investigation, 129(12), 5462-5467.
Jamal I, Kumar V, Vatsa N, Shekhar S, Singh B K, Sharma A, Jana N R (2017) Rescue of altered HDAC activity recovers behavioural abnormalities in a mouse model of Angelman syndrome. Neurobiol Dis 105:99-108.
Jiang, Y. H., Armstrong, D., Albrecht, U., Atkins, C. M., Noebels, J. L., Eichele, G., Beaudet, A. L. (1998). Mutation of the Angelman ubiquitin ligase in mice causes increased cytoplasmic p53 and deficits of contextual learning and long-term potentiation. Neuron, 21(4), 799-811.
Kaphzan H, Hernandez P, Jung J I, Cowansage K K, Deinhardt K, Chao M V., Abel T, Klann E (2012) Reversal of Impaired Hippocampal Long-Term Potentiation and Contextual Fear Memory Deficits in Angelman Syndrome Model Mice by ErbB Inhibitors. Biol Psychiatry 72:182-190.
Mandel-Brehm C, Salogiannis J, Dhamne S C, Rotenberg A, Greenberg M E (2015) Seizure-like activity in a juvenile Angelman syndrome mouse model is attenuated by reducing Arc expression. Proc Natl Acad Sci 112:5129-5134.
Meng L, Person R E, Huang W, Zhu P J, Costa-Mattioli M, Beaudet A L (2013) Truncation of Ube3a-ATS unsilences paternal Ube3a and ameliorates behavioral defects in the Angelman syndrome mouse model. Bartolomei M S, ed. PLoS Genet 9:e1004039.
Paylor R, Nguyen M, Crawley I N, Patrick J, Beaudet A, Orr-Urtreger A (1998) Alpha7 nicotinic receptor subunits are not necessary for hippocampal-dependent learning or sensorimotor gating: a behavioral characterization of Acra7-deficient mice. Learn Mem 5:302-316.
Sonzogni, M., Wallaard, I., Santos, S. S., Kingma, J., du Mee, D., van Woerden, G. M., and Elgersma, Y. (2018). A behavioral test battery for mouse models of Angelman syndrome: a powerful tool for testing drugs and novel Ube3a mutants. Mol. Autism 9, 47.
Steinmetz, A. B., Stern, S. A., Kohtz, A. S., Descalzi, G., and Alberini, C. M. (2018). Insulin-Like Growth Factor II Targets the mTOR Pathway to Reverse Autism-Like Phenotypes in Mice. J. Neurosci. 38, 1015-1029.
Stern, S. A., Kohtz, A. S., Pollonini, G., and Alberini, C. M. (2014). Enhancement of Memories by Systemic Administration of Insulin-Like Growth Factor II. Neuropsychopharmacology 39, 2179-2190.
Sun J, Liu Y, Tran J, O'Neal P, Baudry M, Bi X (2016) mTORC1-S6K1 inhibition or mTORC2 activation improves hippocampal synaptic plasticity and learning in Angelman syndrome mice. Cell Mol Life Sci 73:4303-4314.
Thibert R L, Larson A M, Hsieh D T, Raby A R, Thiele E A (2013) Neurologic Manifestations of Angelman Syndrome. Pediatr Neurol 48:271-279.
Wei, J., Carroll, R. J., Harden, K. K., & Wu, G. (2012). Comparisons of treatment means when factors do not interact in two-factorial studies. Amino Acids, 42, 2031-2035. https://doi.org/10.1007/s00726-011-0924-0.

While the present invention has been described through illustrative embodiments, routine modification will be apparent to those skilled in the art and such modifications are intended to be within the scope of this disclosure.

What is claimed is:

1. A method of preventing, reversing and/or treating seizures in a subject comprising administering to the subject a composition comprising a therapeutically effective amount of IGF-2, or a functional modification or derivative thereof.

2. A method of treating seizures in a subject comprising administering to the subject a composition comprising a therapeutically effective amount of IGF-2, or a functional modification or derivative thereof.

3. The method of claim 1 or 2, wherein the subject has been diagnosed with a neurodevelopmental or neurological disorder.

4. The method of claim 3, wherein the neurodevelopmental or neurological disorder comprises autism spectrum disorder (ASD), Angelman Syndrome (AS), cerebral palsy, Ohtahara Syndrome, benign familial neonatal seizures, West Syndrome, Dravet Syndrome, Rett Syndrome, Tuberous sclerosis, Sturge-Weber Syndrome, Landau-Kleffner Syndrome, Lennox-Gastaut Syndrome, Rasmussen Syndrome, Gelastic Epilepsy, Benign Rolandic Epilepsy, Panayiotopoulos syndrome, Gastaut-type Syndrome, childhood absence epilepsy, or juvenile myoclonic epilepsy.

5. The method of claim 3, wherein the neurodevelopmental or neurological disorder comprises autism spectrum disorder (ASD) or Angelman Syndrome (AS).

6. The method of claim 1 or 2, wherein the subject has a seizure-inducing disease or disorder.

7. The method of claim 6, wherein the seizure-inducing disease or disorder comprises an autoimmune disorder, a cerebral edema, cerebral ischema or hypoxia, head trauma, a central nervous system infection, an intracranial lesion, hyperpyrexia, a metabolic disorder, or a neurocutaneous disorder.

8. The method of claim 1 or 2, wherein the subject has been exposed to a seizure-inducing drug or toxin.

9. The method of claim 1 or 2, wherein the subject has been diagnosed with a congenital abnormality that causes seizures.

10. The method of claim 1 or 2, wherein the subject has been diagnosed with epilepsy.

11. The method of any one of claims 1-10, wherein the seizures comprise focal seizures or generalized seizures.

12. The method of claim 11, wherein the focal seizures comprise focal seizures without loss of consciousness or focal seizures with impaired awareness.

13. The method of claim 11, wherein the generalized seizures comprise absence seizures, tonic seizures, atonic seizures, clonic seizures, myoclonic seizures, or tonic-clonic seizures.

14. The method of any one of claims 1-13, wherein the seizures are audiogenic seizures.

15. The method of any one of claims 1-14, wherein the IGF-2 comprises a sequence of SEQ ID NO: 1 or 3, or a sequence having at least 95% identity thereto.

16. The method of any one of claims 1-15, wherein the IGF-2 comprises a sequence of SEQ ID NO: 11 or 13, or a sequence having at least 95% identity thereto.

17. The method of any one of claims 1-16, wherein treatment comprises alleviating one or more symptoms of epilepsy.

18. The method of any one of claims 1-17, wherein the treatment reduces the severity, duration or frequency of a seizure.

19. The method of any one of claims 1-18, wherein the IGF-2 is administered to the subject in an amount in the range of 1 to 500 μg/kg of the subject's body weight.

20. The method of any one of claims 1-19, wherein the IGF-2 is the only agent in the composition that is capable of binding to IGF-2 receptor.

21. The method of any one of claims 1-20, wherein the composition comprises one or more pharmaceutically acceptable carrier, diluent, or excipient.

22. The method of any one of claims 1-21, where the composition is administered to the subject orally, intranasally, topically, anally, parenterally, intramuscularly, intraperitoneally, intravenously, intracerebral, ocularly, optically, intracerebrally, intraspinally intrathecally, subcutaneously.

23. The use of a composition comprising a therapeutically effective amount of IGF-2, or a functional modification or derivative thereof, in the manufacture of a medicament for the preventing, reversing and/or treating of seizures in a subject.

24. A composition comprising a therapeutically effective amount of IGF-2, or a functional modification or derivative thereof, for use in the prevention, reversion, or treatment of seizures in a subject.

25. The use of claim 23 or 24, wherein the subject has been diagnosed with a neurodevelopmental or neurological disorder.

26. The use of claim 25, wherein the neurodevelopmental or neurological disorder is autism spectrum disorder (ASD), Angelman Syndrome (AS), cerebral palsy, Ohtahara Syndrome, benign familial neonatal seizures, West Syndrome, Dravet Syndrome, Rett Syndrome, Tuberous sclerosis, Sturge-Weber Syndrome, Landau-Kleffner Syndrome, Lennox-Gastaut Syndrome, Rasmussen Syndrome, Gelastic Epilepsy, Benign Rolandic Epilepsy, Panayiotopoulos syndrome, Gastaut-type Syndrome, childhood absence epilepsy, or juvenile myoclonic epilepsy.

27. The use of any one of claims 23-26, wherein the IGF-2 comprises a sequence of SEQ ID NO: 1 or 3, or a sequence having at least 95% identity thereto.

28. The use of any one of claims 23-27, wherein the IGF-2 comprises a sequence of SEQ ID NO: 11 or 13, or a sequence having at least 95% identity thereto.
